(19) United States
(12) Patent Application Publication
(10) Pub. No.: US 2022/0183865 A1
(43) Pub. Date: Jun. 16, 2022

(54) VASCULAR TREATMENT DEVICES AND ASSOCIATED SYSTEMS AND METHODS OF USE

(71) Applicant: The Foundry, LLC, Menlo Park, CA (US)

(72) Inventors: Hanson S. Gifford, III, Woodside, CA (US); Adam Gold, Mountain View, CA (US)

(21) Appl. No.: 17/593,996

(22) PCT Filed: Apr. 1, 2020
(86) PCT No.: PCT/US2020/026278

§ 371 Date: Sep. 29, 2021

Related U.S. Application Data
(60) Provisional application No. 62/287,201, filed on Jan. 26, 2016.

(51) Int. Cl. A61F 2/90

(52) U.S. Cl. CPC A61F 2/90; A61F 2230/001; A61F 2250/0018; A61F 2230/0015; A61F 2230/0008

(57) ABSTRACT

The present technology relates to devices for treating arteries. In several embodiments, for example, the present technology comprises an expandable structure configured to be intravascularly positioned within a lumen of the artery at a treatment site, where the artery has a substantially circular cross-sectional shape at the treatment site prior to deployment of the expandable structure therein. When the expandable structure is in an expanded state and positioned in apposition with the arterial wall at the treatment site under diastolic pressure, the expandable structure may force the artery into a non-circular cross-sectional shape. A cross-sectional area of the artery in the non-circular cross-sectional shape may be less than a cross-sectional area of the artery in the substantially circular cross-sectional shape.

### CROSS-REFERENCE TO RELATED APPLICATION

The present application claims the benefit of U.S. Provisional Application No. 62/827,201, filed Apr. 1, 2019, which is incorporated by reference herein in its entirety.

### TECHNICAL FIELD

The present technology relates to devices for treating blood vessels and associated systems and methods of use. In particular, the present technology is directed to devices for treating arteries.

### BACKGROUND

Aortic elasticity is essential to the healthy function of the heart and circulatory system. As depicted schematically in FIGS. 1A and 1B, healthy large arteries stretch and recoil with the pumping action of the heart, thus serving as elastic reservoirs that enable the arterial tree to undergo large volume changes with little change in pressure. Acting as an elastic buffering chamber behind the heart, the aorta and some of the proximal large vessels store about 50% of the left ventricular stroke volume during systole. In diastole, the elastic forces of the aortic wall forward this 50% of the volume to the peripheral circulation, thus creating a nearly continuous peripheral blood flow. This systolic-diastolic interplay represents the Windkessel function, which has an influence not only on the peripheral circulation but also on the heart, resulting in a reduction of left ventricular afterload and improvement in coronary blood flow and left ventricular relaxation.

Arterial compliance decreases with aging, as well as with pathological changes such as atherosclerosis. Increased aortic stiffness—and the attending loss of Windkessel properties—leads to an increase in systolic blood pressure and a decrease in diastolic blood pressure at any given mean pressure, as well as an increase in left ventricular afterload. For patients suffering from heart failure in which cardiac output is already diminished, sympathetic tone increases to encourage higher blood flow and maintain blood pressure. This further stiffens the aorta, thus placing a greater load on the heart and further decreasing cardiac output. This negative spiral is typically treated with a number of medications to relax the arteries, moderate systolic blood pressure, and encourage greater cardiac output. However, medications often have a limited impact and cause undesirable side-effects.

Therefore, there exists a need for improved therapies for increasing the compliance of the aorta and great vessels.

### SUMMARY

The present technology is directed to devices for increasing arterial compliance and associated systems and methods. According to some embodiments, the device comprises an expandable structure configured to be positioned within the lumen of an artery to influence the cross-sectional shape of the arterial wall during the cardiac cycle. The expandable structure enables the artery to move between a non-circular cross-sectional shape in diastole and a circular (or more circular) cross-sectional shape in systole. In so doing, the expandable structure enables an increase in the cross-sectional area of the artery in response to systolic pressure, thereby providing increased compliance. The expandable structures of the present technology may be particularly beneficial for treating aortic stiffness. For example, the expandable structures of the present technology may be positioned within a substantially inelastic region of the aorta to restore and/or improve the Windkessel function of the aorta during the cardiac cycle. Even without any stretching of the aorta wall itself, the change in arterial volume enabled by the present technology provides significant compliance to the arterial system.

The subject technology is illustrated, for example, according to various aspects described below, including with reference to FIGS. 2A-22B. Various examples of aspects of the subject technology are described as numbered clauses (1, 2, 3, etc.) for convenience. These are provided as examples and do not limit the subject technology.
1. A device for treating an artery, the artery having a circular cross-sectional shape, the device comprising:
   an expandable, generally tubular mesh configured to be intravascularly positioned within a lumen of the artery at a treatment site, the mesh being transformable between a low-profile state for delivery to the treatment site and an expanded state in which the mesh has a non-circular cross-sectional shape,
   wherein the mesh is configured to expand into apposition with the arterial wall at the treatment site, thereby increasing the radius of curvature of opposing portions of the wall such that the wall assumes the non-circular cross-sectional shape of the mesh,
   wherein (a) under diastolic pressure, the mesh holds the arterial wall in the non-circular cross-sectional shape, (b) the mesh allows the wall to deform in response to systolic pressure such that the wall assumes a second cross-sectional shape in which a distance between the opposing portions of the wall increases relative to the distance when the wall is in the non-circular cross-sectional shape, and (c) a cross-sectional area of the mesh tube in the second cross-sectional shape is greater than a cross-sectional area of the mesh in the non-circular cross-sectional shape, thereby increasing compliance of the artery.
2. The device of Clause 1, wherein the mesh is configured absorb and reduce the energy transmitted to the arterial system by the left ventricle during systole.
3. The device of any one of the preceding Clauses, wherein the mesh remains in contact with the opposing portions of the arterial wall as the wall deforms from the non-circular cross-sectional shape to the second cross-sectional shape.
4. The device of any one of the preceding Clauses, wherein, during diastole, the mesh forces the opposing portions of the arterial wall to have a radius of curvature that is greater than a radius of curvature of the opposing portions during systole.
5. The device of any one of the preceding Clauses, wherein the mesh remains in direct contact with an inner surface of the arterial wall throughout a full cardiac cycle.
6. The device of any one of the preceding Clauses, wherein the mesh remains in direct, substantially continuous circumferential contact with an inner surface of the arterial wall throughout a full cardiac cycle.
7. The device of any one of the preceding Clauses, wherein the mesh is configured to expand into contact with the arterial wall without significantly stretching the wall.
8. The device of any one of the preceding Clauses, wherein the non-circular shape is an oval.
9. The device of the preceding Clause, wherein a cross-sectional area of the cross-sectional shape is defined by a major diameter and a minor diameter.
10. The device of the preceding Clause, wherein the minor diameter is about 6 mm to about 12 mm, and the major diameter is about 15 mm to about 40 mm.
11. The device of any one Clauses 1 to 7, wherein the non-circular shape is a rhomboid.
12. The device of any one of the preceding Clauses, wherein, in a relaxed state, opposing sidewalls of the mesh are generally flat such that, at least during diastole, the opposing portions of the arterial wall conform to the generally flat opposing sidewalls of the mesh.
13. The device of any one of the preceding Clauses, wherein, in a relaxed state, opposing sidewalls of the mesh are generally flat and wherein, (a) during diastole, the opposing portions of the arterial wall conform to and maintain apposition with the generally flat opposing sidewalls of the mesh, and (b) the opposing sidewalls of the mesh remain generally flat during systole such that the opposing portions of the arterial wall in apposition with the generally flat sidewalls also remain generally flat during systole.
14. The device of any one of the preceding Clauses, wherein a sidewall of the mesh has generally straight portions connected by curved portions, and wherein the mesh preferentially flexes at the curved portions during systole such that the generally straight portions remain straight during systole.
15. The device of Clause 14, wherein in its relaxed state the mesh has a generally rhomboid shape with two opposed acute curves at the major diameter of the mesh, and two obtuse angles at the minor diameter of the mesh. 16. The device of Clause 14 or Clause 15, wherein a radius of curvature of the acutely curved portions increases in response to forces from the arterial wall during systole.
17. The device of any one of Clauses 14 to 16, wherein, at least when the mesh is in a relaxed state, the generally straight portions of the sidewalls are generally parallel to one another.
18. The device of any one of Clauses 14 to 17, wherein the mesh comprises two generally straight portions, three generally straight portions, four generally straight portion, five generally straight portions, or six generally straight portions.
19. The device of any one of the preceding Clauses, wherein, when implanted within the arterial lumen, the device is configured to decrease systolic pressure and increase diastolic pressure.
20. The device of any one of the preceding Clauses, wherein, when implanted within the arterial lumen, the device is configured to increase a compliance of the artery without substantially stretching the arterial wall.
21. The device of any of the preceding Clauses, wherein the mesh is configured absorb energy transmitted by a pulse wave, thereby increasing compliance of the arterial system relative to arterial compliance without the mesh implanted within the artery.
22. A device for treating an artery, the artery having a generally circular cross-sectional shape, the device comprising:
   a device configured to be intravascularly positioned within a lumen of the artery at a treatment site, the device being transformable between a low-profile state for delivery to the treatment site and an expanded state after delivery,
   wherein the device is configured to expand into apposition with the arterial wall at the treatment site and change the cross-sectional shape of the artery to decrease a cross-sectional area of the artery in diastole relative to a cross-sectional area of the artery in diastole without the device positioned therein,
   wherein the device elastically deforms under systolic pressure to allow an increase in the cross-sectional area of the artery, thereby increasing compliance of the artery.
23. A device of any one of the preceding Clauses, wherein a spring constant of the device is configured to allow the artery to deform the stent towards a more circular cross-sectional shape during systole.
24. The device of any one of the preceding Clauses, wherein the mesh comprises a generally tubular sidewall defining a lumen therethrough, wherein the sidewall comprises a plurality of strut sections and a plurality of bridge sections, wherein: (a) each of the strut sections extends circumferentially about the mesh and comprises a plurality of struts, and (b) each of the bridge sections extends between adjacent strut sections and comprises at least one bridge.
25. The device of Clause 24, wherein the struts within each of the strut sections are connected to each other end-to-end in a zig-zag configuration forming a circumferential band.
26. The device of Clause 25, wherein the strut sections in certain first areas of the circumference have different dimensions or shapes than certain second areas of the circumference, leading to higher deformation of the struts in the first areas relative to the deformation of the struts in the second areas.
27. The device of any one of Clauses 24 to 26, wherein a majority of the strut sections along a length of the mesh maintain substantially continuous circumferential contact with the arterial wall during a full cardiac cycle.
28. The device of any one of Clauses 24 to 27, wherein the strut sections along at least 80% of the length of the mesh maintain substantially continuous circumferential contact with the arterial wall during a full cardiac cycle.
29. The device of any one of Clauses 24 to 28, wherein the strut sections along at least 90% of the length of the mesh maintain substantially continuous circumferential contact with an inner surface of the arterial wall during a full cardiac cycle.
30. The device of any one of Clauses 24 to 29, wherein the strut sections along at least 95% of the length of the mesh maintain substantially continuous circumferential contact with an inner surface of the arterial wall during a full cardiac cycle.
31. The device of any one of Clauses 24 to 30, wherein a longitudinal distance of each strut section is of from about 5 mm to about 15 mm.
32. The device of any one of the preceding Clauses, wherein at least a portion of the mesh is configured to promote tissue ingrowth around the mesh.
33. The device of any one of the preceding Clauses, further comprising a coating along all or a portion of the mesh to promote tissue ingrowth around the mesh.
34. The device of any one of the preceding Clauses, wherein a surface of the mesh is textured to promote tissue ingrowth.
35. The device of any one of the preceding Clauses, further comprising a material coupled to the mesh that promotes tissue ingrowth.
36. The device of any one of the preceding Clauses, wherein the artery is a portion of the aorta.
37. The device of any one of the preceding Clauses, wherein at least a portion of the device is configured to be positioned at a treatment site along an ascending aorta.
38. The device of any one of the preceding Clauses, wherein at least a portion of the device is configured to be positioned at a treatment site along an aortic arch.
39. The device of any one of the preceding Clauses, wherein at least a portion of the device is configured to be positioned at a treatment site along a descending thoracic aorta.
40. The device of any one of the preceding Clauses, wherein at least a portion of the device is configured to be positioned at a treatment site along an abdominal aorta.
41. The device of any one of the preceding Clauses, wherein at least a portion of the device is configured to be positioned at a treatment site along an iliac artery.
42. The device of any one of the preceding Clauses, wherein the device is configured to be positioned at a treatment site within at least one of a left common carotid artery, a right common carotid artery, and a brachiocephalic artery.
43. The device of any one of the preceding Clauses, wherein the device is configured to treat heart failure.
44. The device of any one of the preceding Clauses, wherein a cross-sectional shape of the mesh becomes more circular towards one or both ends of the mesh.
45. The device of any one of the preceding Clauses, further comprising a radiopaque material.
46. The device of any one of the preceding Clauses, wherein the device includes one or more radiopaque markers coupled to the mesh.
47. The device of any one of the preceding Clauses, wherein the device includes first and second radiopaque markers at distinct first and second locations along the mesh, and wherein the first and second locations represent portions of the mesh configured to be positioned at anterior and posterior positions, respectively, when the device is implanted.
48. The device of any one of the preceding Clauses, wherein all or a portion of the mesh includes an anti-proliferative coating.
49. The device of any one of the preceding Clauses, wherein all or a portion of the mesh includes an anti-thrombotic coating.
50. The device of any one of the preceding Clauses, wherein the mesh is self-expanding.
51. The device of any one of the preceding Clauses, wherein the mesh is a laser-cut stent.
52. The device of any one of the preceding Clauses, wherein the mesh comprises a stent cut from a tube of superelastic material such as Nitinol.
53. The device of any one of the preceding Clauses, wherein the mesh comprises a stent formed from stainless steel or cobalt-chromium wires which allow elastic deformation from a low-profile shape for delivery to an expanded shape after delivery.
54. The device of any one of the preceding Clauses, wherein the mesh is a braid.
55. A device for treating an artery, the artery having a circular cross-sectional shape, the device comprising:
   an expandable mesh configured to be intravascularly positioned within a lumen of the artery at a treatment site, the mesh comprising a tubular sidewall transformable between a low-profile state for delivery to the treatment site and an expanded state in which the sidewall has (a) a non-circular cross-sectional shape, and (b) alternating first and second portions about its circumference, wherein each of the first portions have a first radius of curvature and each of the second portions have a second radius of curvature smaller than the first radius of curvature,
   wherein, when deployed with the arterial lumen, the arterial wall conforms to the shape of the mesh, and
   wherein the mesh has (a) a chronic outward force great enough to hold the arterial wall in the non-circular cross-sectional shape under diastolic pressure, and (b) a radial resistive force low enough such that, during systole, the forces applied to the mesh by the arterial wall urge the first portions of the sidewall away from one another and the second portions of the sidewall towards one another such that the mesh assumes a second cross-sectional shape having an area greater than an area of the diastolic non-circular cross-sectional shape.
56. The device of Clause 55, wherein the mesh is configured absorb and reduce the energy transmitted to the arterial system by the left ventricle during systole.
57. The device of Clause 55 or Clause 56, wherein the arterial wall remains apposed to the sidewall of the mesh in both the non-circular shape and the second shape.
58. The device of any one of the preceding Clauses, wherein the mesh is configured to heal into the arterial wall such that the arterial wall adapts the circumference of the mesh.
59. The device of any one of the preceding Clauses, wherein the mesh preferentially flexes more at the second portions during systole than the first portions such that difference between the first and second radii of curvature decreases.
60. The device of any one of the preceding Clauses, wherein the first portions of the mesh are generally straight when the mesh is in a relaxed state.
61. The device of Clause 60, wherein the first portions of the mesh remain generally straight even when the mesh is implanted within the arterial lumen and under the forces from the arterial wall during systole.
62. The device of Clause 60 or Clause 61, wherein, at least when the mesh is in a relaxed state, the generally straight portions of the sidewalls are generally parallel to one another.
63. The device of any one of Clauses 60 to 62, wherein the mesh comprises two generally straight portions, three generally straight portions, four generally straight portion, five generally straight portions, or six generally straight portions.
64. The device of any one of the preceding Clauses, wherein the sidewall comprises a plurality of strut sections and a plurality of bridge sections, wherein: (a) each of the strut sections extend circumferentially about the mesh and comprise a plurality of struts, and (b) each of the bridge sections extend between adjacent strut sections and comprise at least one bridge.
65. The device of Clause 64, wherein the struts within each of the strut sections are connected to each other end-to-end in a zig-zag configuration.
66. The device of Clause 64 or Clause 65, wherein a majority of the strut sections along a length of the mesh maintain substantially continuous circumferential contact with the arterial wall during a full cardiac cycle.
67. The device of any one of Clauses 64 to 66, wherein the strut sections along at least 80% of the length of the mesh maintain substantially continuous circumferential contact with the arterial wall during a full cardiac cycle.
68. The device of any one of Clauses 64 to 67, wherein the strut sections along at least 90% of the length of the mesh maintain substantially continuous circumferential contact with an inner surface of the arterial wall during a full cardiac cycle.
69. The device of any one of Clauses 64 to 68, wherein the strut sections along at least 95% of the length of the mesh maintain substantially continuous circumferential contact with an inner surface of the arterial wall during a full cardiac cycle.
70. The device of any one of Clauses 64 to 69, wherein a longitudinal distance of each strut section is of from about 5 mm to about 15 mm.
71. The device of any one of the preceding Clauses, wherein the non-circular shape is an oval.
72. The device of the preceding Clause, wherein a cross-sectional area of the cross-sectional shape is defined by a major diameter and a minor diameter.
73. The device of the preceding Clause, wherein the minor diameter is about 6 mm to about 12 mm, and the major diameter is about 20 mm to about 40 mm.
74. The device of any one Clauses 1 to 70, wherein the non-circular shape is a rhomboid.
75. The device of the preceding Clause, wherein the relaxed distance between two opposing apices of the rhomboid shape is between 6 mm and 12 mm, and the distance between the other two opposing apices is between 20 mm and 40 mm.
76 The device of either of the two preceding Clauses, wherein the rhomboid shape is designed to be more flexible around the curved apices, and less flexible along the generally straight sections.
77. The device of any one of the preceding Clauses, wherein the device is between 100 mm and 200 mm in length.
78. The device of any one of the preceding Clauses, wherein at least a portion of the mesh is configured to promote tissue ingrowth around the mesh.
79. The device of any one of the preceding Clauses, further comprising a coating along all or a portion of the mesh to promote tissue ingrowth around the mesh.
80. The device of any one of the preceding Clauses, wherein a surface of the mesh is textured to promote tissue ingrowth.
81. The device of any one of the preceding Clauses, further comprising a material coupled to the mesh that promotes tissue ingrowth.
82. The device of any one of the preceding Clauses, wherein the artery is a portion of the aorta.
83. The device of any one of the preceding Clauses, wherein the device is configured to be positioned at a treatment site along an ascending aorta.
84. The device of any one of the preceding Clauses, wherein the device is configured to be positioned at a treatment site along an aortic arch.
85. The device of any one of the preceding Clauses, wherein the device is configured to be positioned at a treatment site along a descending thoracic aorta.
86. The device of any one of the preceding Clauses, wherein the device is configured to be positioned at a treatment site along an abdominal aorta.
87. The device of any one of the preceding Clauses, wherein the device is configured to be positioned at a treatment site along an iliac artery.
88. The device of any one of the preceding Clauses, wherein the device is configured to be positioned at a treatment site within at least one of a left common carotid artery, a right common carotid artery, and a brachiocephalic artery.
89. The device of any one of the preceding Clauses, wherein the device is configured to treat heart failure.
90. The device of any one of the preceding Clauses, wherein the mesh remains in direct, substantially continuous circumferential contact with an inner surface of the arterial wall throughout a full cardiac cycle.
91. The device of any one of the preceding Clauses, wherein the mesh is configured to expand into contact with the arterial wall without significantly stretching the wall.
92. The device of any one of the preceding Clauses, wherein a cross-sectional shape of the mesh becomes more circular towards one or both ends of the mesh.
93. The device of any one of the preceding Clauses, further comprising a radiopaque material.
94. The device of any one of the preceding Clauses, wherein the device includes one or more radiopaque markers coupled to the mesh.
95. The device of any one of the preceding Clauses, wherein the device includes first and second radiopaque markers at distinct first and second locations along the mesh, and wherein the first and second locations represent portions of the mesh configured to be positioned at anterior and posterior positions, respectively, when the device is implanted.
96. The device of any one of the preceding Clauses, wherein all or a portion of the mesh includes an anti-proliferative coating.
97. The device of any one of the preceding Clauses, wherein all or a portion of the mesh includes an anti-thrombotic coating.
98. The device of any one of the preceding Clauses, wherein the mesh is self-expanding.
99. The device of any one of the preceding Clauses, wherein the mesh is a laser-cut stent.
100. The device of any one of the preceding Clauses, wherein the mesh is a braid.
101. The device of any one of the preceding Clauses, wherein the mesh is configured absorb energy transmitted by a pulse wave, thereby reducing stress on the arterial wall relative to a stress on the arterial wall without the mesh implanted within the artery.
102. A method for treating heart failure, the method comprising:
   positioning a device within an artery, the device imparting a non-circular cross-sectional shape to that artery in diastole to reduce its cross-sectional area,
   wherein during systole, the force of blood pressure within that artery overcomes the shape change imparted by the device and allows the artery to assume a second, more circular cross-sectional shape with greater cross-sectional area,
   thereby increasing the compliance of the arterial system.
103. A method for treating an artery of a patient, the method comprising:
   positioning a generally tubular mesh in apposition with the wall of the artery, the mesh having a non-circular cross-sectional shape,
   wherein during diastole, the mesh holds the artery in the non-circular shape;
   and during systole, the mesh allows the artery to be urged into a second cross-sectional shape in response to systolic pressure, wherein the second cross-sectional shape is generally more circular and has a greater cross-sectional area,
   thereby increasing a compliance of the artery.
104. A method for treating an artery of a patient, the method comprising:
   positioning a generally tubular mesh in apposition with the wall of the artery, the mesh having (a) a non-circular cross-sectional shape, and (b) alternating first and second portions about its circumference, wherein each of the first portions have a first radius of curvature and each of the second portions have a second radius of curvature greater than the first radius of curvature,
   during diastole, holding the artery in the non-circular shape of the mesh while maintaining apposition between the arterial wall and the mesh;
   during systole, allowing the mesh to be urged into a second cross-sectional shape by the artery in response to systolic pressure, wherein the forces applied to the mesh by the arterial wall urge the first portions of a mesh sidewall away from one another and the second portions of the sidewall towards one another; and
   increasing a compliance of the artery.
105. The method of any one of the preceding Clauses, wherein an area of the second cross-sectional shape is greater than an area of the non-circular cross-sectional shape.
106. The method of any one of the preceding Clauses, wherein the mesh maintains substantially continuous apposition with a full circumference of the arterial wall during diastole and systole.
107. The method of any one of the preceding Clauses, further comprising promoting tissue ingrowth with the mesh.
108. The method of any one of the preceding Clauses, further comprising absorbing, with the implanted mesh, at least a portion of the energy of systolic pressure and volume.
109. The method of any one of the preceding Clauses, further comprising reducing stress on the arterial wall during the cardiac cycle relative to stress on the arterial wall during the cardiac cycle without the implanted mesh.
110. The method of any one of the preceding Clauses, further comprising, in response to systolic pressure, decreasing a radius of curvature of opposing portions of the arterial wall in apposition with the second portions of the mesh.
111. The method of Clause 110, wherein, the respective radii of curvature of opposing portions of the arterial wall in apposition with the first portions of the mesh remain generally constant while the opposing portions in apposition with the second portions of the mesh changes.
112. The method of any one of the preceding Clauses, further comprising, in response to systolic pressure, increasing a radius of curvature of opposing portions of the arterial wall in apposition with the first portions of the mesh.
113. The method of any one of the preceding Clauses, further comprising substantially flattening at least a portion of the arterial wall.
114. The method of any one of the preceding Clauses, wherein the mesh covers at least 100 mm of length of the artery.
115. The method of any one of the preceding Clauses, wherein intravascularly positioning a mesh includes intravascularly positioning the mesh within the aortic arch.
116. The method of any one of the preceding Clauses, wherein intravascularly positioning a mesh includes intravascularly positioning the mesh within the ascending aorta.
117. The method of any one of the preceding Clauses, wherein intravascularly positioning a mesh includes intravascularly positioning the mesh within the thoracic aorta.
118. The method of any one of the preceding Clauses, wherein intravascularly positioning a mesh includes intravascularly positioning the mesh within the abdominal aorta.
119. The method of any one of the preceding Clauses, wherein intravascularly positioning a mesh includes intravascularly positioning the mesh within an iliac artery.
120. The method of any one of the preceding Clauses, wherein intravascularly positioning a mesh includes intravascularly positioning the mesh within at least one of a left common carotid artery, a right common carotid artery at a treatment site.
121. The method of any one of the preceding Clauses, wherein positioning the mesh in apposition with the wall of the artery includes expanding the mesh with a balloon.
122. The method of any one of the preceding Clauses, wherein positioning the mesh in apposition with the wall of the artery includes withdrawing a sheath to expose the mesh to allow the mesh to self-expand.
123. The method of any one of the preceding Clauses, wherein:
   the mesh is a first mesh,
   the first mesh is intravascularly positioned at a first arterial location, and
   the method further comprises intravascularly positioning a second mesh at a second arterial location different than the first arterial location.
124. The method of any one of the preceding Clauses, further comprising increasing a diastolic pressure of the patient.
125. The method of any one of the preceding Clauses, further comprising decreasing a systolic pressure of the patient.
126. The method of any one of the preceding Clauses, further comprising both increasing a diastolic blood pressure and decreasing a systolic blood pressure of the patient.
127. The method of any one of the preceding Clauses, further comprising improving the Windkessel function of the aorta.
128. A device for treating an artery of a human patient, the device comprising:
   an expandable structure configured to be intravascularly positioned within a lumen of the artery at a treatment site, wherein the artery has a substantially circular cross-sectional shape at the treatment site prior to deployment of the expandable structure therein, and wherein, when the expandable structure is in an expanded state and positioned in apposition with the arterial wall at the treatment site under diastolic pressure, the expandable structure forces the artery into a non-circular cross-sectional shape, wherein a cross-sectional area of the artery in the non-circular cross-sectional shape is less than a cross-sectional area of the artery in the substantially circular cross-sectional shape.
129. The device of Clause 128, wherein, when the expandable structure is in the expanded state and in apposition with the arterial wall at the treatment site under systolic pressure, the arterial wall deforms in response to the increase in blood pressure towards a more circular cross-sectional shape, thereby deforming the expandable structure as well.
130. The device of Clause 129, wherein a cross-sectional area of the artery in the more circular cross-sectional shape is greater than a cross-sectional area of the artery in the non-circular cross-sectional shape.
131. The device of any one of Clauses 128 to 130, wherein the non-circular cross-sectional shape is one of an oval, an ellipse, a rhomboid, or an hourglass.
132. The device of any one of Clauses 128 to 131, wherein the expandable structure comprises two relatively rigid linear elements with curved cross-sections, separated by one or more springs which hold them apart.
133. The device of Clause 132, wherein a preload and a geometry of the springs cause a force holding the linear elements apart to decrease as the two linear elements are pressed closer together.
134. The device of any one of claims 128 to 133, wherein the artery is the aorta.
135. The device of any one of claims 128 to 134, wherein the expandable structure comprises a mesh.
136. The device of any one of claims 128 to 135, wherein the expandable structure comprises a self-expanding mesh.
137. The device of any one of claims 128 to 136, wherein the expandable structure comprises a stent formed of a plurality of interconnected struts forming a plurality of cells therebetween.
138. The device of any one of claims 128 to 136, wherein the expandable structure comprises an expandable braid.
139. The device of any one of claims 128 to 138, wherein the expandable structure comprises a superelastic material.
140. The device of any one of claims 128 to 139, wherein the expandable structure is non-circular in the expanded state.
141. The device of any one of claims 128 to 140, wherein the expandable structure is non-circular when positioned in the arterial lumen in the expanded state.
142. A device for treating an artery, the device comprising:
   an expandable structure configured to be intravascularly positioned within a lumen of the artery at a treatment site, the expandable structure being generally tubular and movable between a low-profile state for delivery to the treatment site and an expanded state in which the expandable structure has a first cross-sectional shape, the first cross-sectional shape having a long dimension and a short dimension orthogonal to the long dimension, wherein the expandable structure comprises first portions at either side of the long dimension and second portions at either side of the short dimension,
      wherein—
      when the expandable structure is deployed within the arterial lumen, the arterial wall conforms to a shape of the expandable structure, and
      under diastolic pressure, the expandable structure assumes the first cross-sectional shape and forces the artery into the first cross-sectional shape, wherein a cross-sectional area of the artery in the first cross-sectional shape is less than a cross-sectional area of the artery prior to deployment of the expandable structure therein.
143. The device of claim 142, wherein, in response to forces exerted on the expandable structure by the arterial wall under systolic pressure, the first portions move towards one another along the long dimension and the second portions move away from one another along the short dimension such that the expandable structure and the artery move toward a second cross-sectional shape having a cross-sectional area greater than a cross-sectional area of the first cross-sectional shape.
144. The device of claim 142 or claim 143, wherein a circumference of the sidewall when in the first cross-sectional shape is approximately the same as a circumference of the sidewall when in the second cross-sectional shape.
145. The device of any one of claims 142 to 144, wherein a circumference of the artery before the expandable structure is positioned therein is substantially the same as a circumference of the artery when the expandable structure is expanded therein.
146. The device of any one of claims 142 to 146, wherein the sidewall includes a plurality of bend regions along which the sidewall is configured to preferentially bend as it moves between the first and second cross-sectional shapes.
147. The device of any one of claims 142 to 147, wherein the sidewall includes one of the bend regions at each of the first portions and at each of the second portions.
148. The device of any one of claims 142 to 148, wherein the first cross-sectional shape is non-circular.
149. The device of any one of claims 142 to 148, wherein the second cross-sectional shape is substantially circular.
150. The device of any one of claims 142 to 149, wherein the non-circular cross-sectional shape is one of a rhomboid, an oval, an ellipse, or an hourglass.
151. The device of any one of claims 142 to 150, further comprising a first support proximate one of the first portions and a second support proximate the other one of the first portions, wherein the first and second supports are configured to engage an opposing portion of the sidewall and/or a support extending from the opposing portion of the sidewall to prevent the short dimension of the expandable structure from falling below a minimum distance.
152. The device of any one of claims 142 to 151, wherein the artery is the aorta.
153. The device of any one of claims 142 to 152, wherein the expandable structure comprises a mesh.
154. The device of any one of claims 142 to 153, wherein the expandable structure comprises a self-expanding mesh.
155. The device of any one of claims 142 to 154, wherein the expandable structure comprises a stent formed of a plurality of interconnected struts forming a plurality of cells therebetween.
156. The device of any one of claims 142 to 155, wherein the expandable structure comprises an expandable braid.
157. The device of any one of claims 142 to 156, wherein the expandable structure comprises a superelastic material.
158. A device for treating an artery of a human patient, the device comprising:
   an expandable structure configured to be intravascularly positioned within a lumen of the artery at a treatment site, the expandable structure comprising a tubular sidewall defining a lumen therethrough, the sidewall forming a non-circular cross-sectional shape when the expandable structure is in a relaxed state, wherein the sidewall comprises:
      a long dimension and a short dimension orthogonal to the long dimension, first and second resilient bend regions at either side of the short dimension, and
      wherein, when the expandable structure is in the relaxed state, each of the first and second bend regions are biased towards the lumen such that each of the first and second bend regions exert a spring force that is generally constant when the sidewall is compressed along the long dimension.
159. The device of claim 158, wherein the first and second bend regions are convex towards the lumen.
160. The device of claim 158 or claim 159, further comprising third and fourth resilient bend regions at either side of the long dimension, wherein the third and fourth bend regions are concave towards the lumen.
161. The device of any one of claims 158 to 160, wherein the first and second bend regions are convex towards the lumen, and wherein the device further comprises third and fourth resilient bend regions at either side of the long dimension that are concave towards the lumen.
162. The device of any one of claims 158 to 161, further comprising third and fourth resilient bend regions at either side of the long dimension, wherein one or both of the third and fourth bend regions are preloaded.
163. The device of any one of claims 158 to 162, further comprising:
   third and fourth resilient bend regions at either side of the long dimension,
   a first support proximate the third bend region and a second support proximate the fourth bend region, wherein the first and second supports are configured to extend into the lumen to prevent the short dimension of the expandable structure from decreasing below a minimum distance.
164. The device of any one of claims 158 to 163, wherein the non-circular cross-sectional shape is one of an oval, an ellipse, a rhomboid, or an hourglass.
165. The device of any one of claims 158 to 164, wherein the artery is the aorta.
166. The device of any one of claims 158 to 165, wherein the expandable structure comprises a mesh.
167. The device of any one of claims 158 to 166, wherein the expandable structure comprises a self-expanding mesh.
168. The device of any one of claims 158 to 167, wherein the expandable structure comprises a stent formed of a plurality of interconnected struts forming a plurality of cells therebetween.
169. The device of any one of claims 158 to 167, wherein the expandable structure comprises an expandable braid.
170. The device of any one of claims 158 to 169, wherein the expandable structure comprises a superelastic material.
171. A device for treating an artery of a human patient, the device comprising:
   an expandable structure configured to be intravascularly positioned within a lumen of the artery at a treatment site, the expandable structure comprising a tubular sidewall defining a lumen therethrough, the sidewall forming a non-circular cross-sectional shape when the expandable structure is in a relaxed state, and wherein the cross-sectional shape comprises:
      a long dimension and a short dimension orthogonal to the long dimension,
      first, second, third, and fourth resilient bend regions spaced apart along a circumference of the cross-sectional shape, wherein the first and third bend regions are disposed at either side of the short dimension and the second and fourth bend regions are disposed at either side of the long dimension, the second and fourth bend regions forming respective second and fourth internal angles, and
      wherein, when the expandable structure is in the relaxed state, each of the first and third bend regions are preloaded such that, as the second and fourth angles increase, the first and third bend regions have an initial force resisting moving away from one another.
172. The device of claim 171, wherein the first and third bend regions are convex towards the lumen.
173. The device of claim 171 or claim 172, wherein the second and fourth bend regions are concave towards the lumen.
174. The device of any one of claims 171 to 173, wherein the artery is the aorta.
175. The device of any one of claims 171 to 174, wherein the expandable structure comprises a mesh.
176. The device of any one of claims 171 to 175, wherein the expandable structure comprises a self-expanding mesh.
177. The device of any one of claims 171 to 176, wherein the expandable structure comprises a stent formed of a plurality of interconnected struts forming a plurality of cells therebetween.
178. The device of any one of claims 171 to 176, wherein the expandable structure comprises an expandable braid.
179. The device of any one of claims 171 to 178, wherein the expandable structure comprises a superelastic material.
180. The device of any one of the preceding Clauses, wherein at least a portion of the mesh is configured to promote tissue ingrowth around the mesh.
181. The device of any one of the preceding Clauses, further comprising a coating along all or a portion of the mesh to promote tissue ingrowth around the mesh.
182. The device of any one of the preceding Clauses, wherein a surface of the mesh is textured to promote tissue ingrowth.
183. The device of any one of the preceding Clauses, further comprising a material coupled to the mesh that promotes tissue ingrowth.
184. The device of any one of the preceding Clauses, wherein the artery is a portion of the aorta.
185. The device of any one of the preceding Clauses, wherein at least a portion of the device is configured to be positioned at a treatment site along an ascending aorta.
186. The device of any one of the preceding Clauses, wherein at least a portion of the device is configured to be positioned at a treatment site along an aortic arch.
187. The device of any one of the preceding Clauses, wherein at least a portion of the device is configured to be positioned at a treatment site along a descending thoracic aorta.
188. The device of any one of the preceding Clauses, wherein at least a portion of the device is configured to be positioned at a treatment site along an abdominal aorta.
189. The device of any one of the preceding Clauses, wherein at least a portion of the device is configured to be positioned at a treatment site along an iliac artery.
190. The device of any one of the preceding Clauses, wherein the device is configured to be positioned at a treatment site within at least one of a left common carotid artery, a right common carotid artery, and a brachiocephalic artery.
191. The device of any one of the preceding Clauses, wherein the device is configured to treat heart failure.
192. The device of any one of the preceding Clauses, wherein a cross-sectional shape of the mesh becomes more circular towards one or both ends of the mesh.
193. The device of any one of the preceding Clauses, further comprising a radiopaque material.
194. The device of any one of the preceding Clauses, wherein the device includes one or more radiopaque markers coupled to the expandable structure.

### BRIEF DESCRIPTION OF THE DRAWINGS

Many aspects of the present disclosure can be better understood with reference to the following drawings. The components in the drawings are not necessarily to scale. Instead, emphasis is placed on illustrating clearly the principles of the present disclosure.

FIGS. 1A and 1B are conceptual diagrams demonstrating arterial compliance during the cardiac cycle.

FIGS. 2A and 2B schematically depict a test setup for estimating the forces required to change the cross-sectional shape of an aorta from a circle to an ellipse.

FIG. 3 is a plot of major diameter versus force per linear inch obtained using the test setup of FIGS. 2A and 2B.

FIGS. 4A and 4B schematically depict a test setup for estimating the forces exerted by an ovular stent on the surrounding aorta when the stent is compressed along its major axis.

FIG. 5 is a plot of major diameter versus force per linear inch obtained using the test setup of FIGS. 4A and 4B. In FIG. 5, the plot is shown superimposed on the plot of FIG. 3.

FIG. 6 is a plot of major diameter versus force per linear inch obtained using the test setup of FIGS. 2A and 2B. In FIG. 6, the plot is shown superimposed on the plot of FIG. 3.

FIG. 7A is a side view of a mesh configured in accordance with several embodiments of the present technology.

FIG. 7B is a cross-sectional end view of the mesh shown in FIG. 7A, taken along line 7B-7B.

FIG. 7C is an enlarged, isolated view of a strut of the device shown in FIG. 7A.

FIG. 7D is an enlarged, isolated view of a strut of the device shown in FIG. 7B.

FIGS. 8A and 8B show the device of FIGS. 7A and 7B positioned within an artery during systole and diastole, respectively, in accordance with several embodiments of the present technology.

FIGS. 9A and 9B depict a method for forming a preloaded device in accordance with several embodiments of the present technology.

FIGS. 10A-10D depict a method for forming a preloaded device in accordance with several embodiments of the present technology.

FIGS. 11A and 11B depict a method for forming a preloaded device in accordance with several embodiments of the present technology.

FIGS. 12A-12F are end views of several devices of the present technology that have different cross-sectional shapes.

FIGS. 13A and 13B are an end view and a side view, respectively, of a device configured in accordance with several embodiments of the present technology.

FIGS. 14A-14D are end views of several devices of the present technology having different supports.

FIG. 15A is a side view of a device configured in accordance with several embodiments of the present technology.

FIG. 15B is an axial cross-sectional view of the device shown in FIG. 15A taken along line 15B-15B.

FIG. 15C is an axial cross-sectional view of the device shown in FIG. 15A taken along line 15B-15B.

FIG. 15D is an axial cross-sectional view of the device shown in FIG. 15A taken along line 15B-15B.

FIG. 16 is an isometric view of a device configured in accordance with several embodiments of the present technology.

FIGS. 17A-17D show examples of different cross-sectional shapes for the device of FIG. 16.

FIG. 18A-18E show examples of different cross-sectional shapes for a non-circumferential device configured to apply force to two opposing walls of the aorta.

FIG. 19 is an isometric view of a non-circumferential device configured in accordance with several embodiments of the present technology.

FIGS. 20A-20C depict a portion of a device comprising a continuous wire configured in accordance with several embodiments of the present technology.

FIGS. 21A and 21B are cross-sectional shapes of a device at different blood pressures configured in accordance with several embodiments of the present technology.

FIGS. 22A and 22B show cross-sectional views of delivery balloons configured in accordance with several embodiments of the present technology.

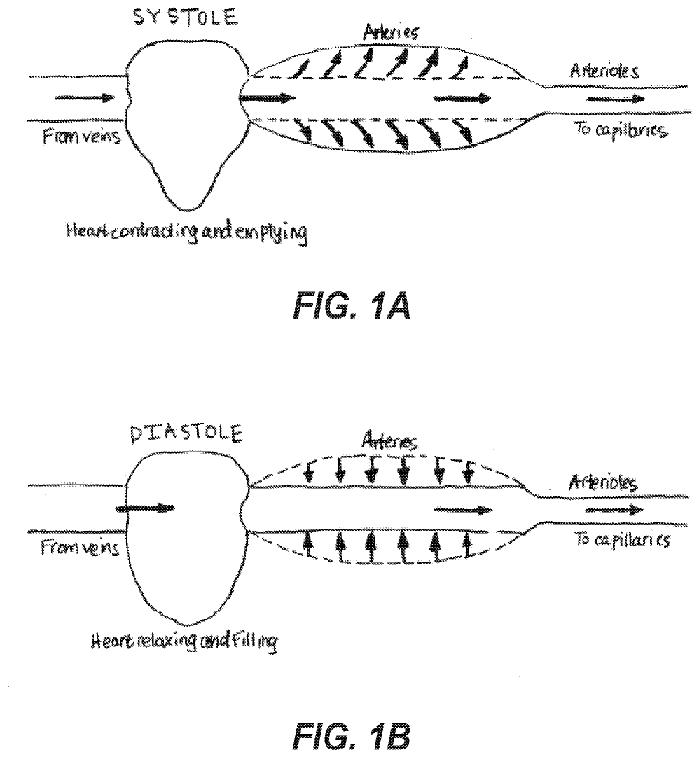

### DETAILED DESCRIPTION

The present technology relates to devices, systems, and methods for treating blood vessels. According to some embodiments, the device comprises an expandable structure configured to be positioned within the lumen of an artery to influence the cross-sectional shape of the arterial wall during the cardiac cycle. Under diastolic pressure, the expandable structure exerts an elongating force on the arterial wall sufficient to deform the arterial wall into a cross-sectional shape having a cross-sectional area that is less than the natural cross-sectional area of the artery during diastole. The elongating force exerted by the expandable structure, however, may be low enough such that under systolic pressure, the expandable structure allows the artery to deform into a more circular cross-sectional shape.

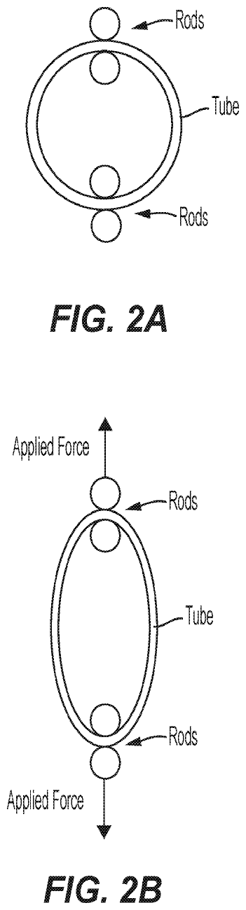

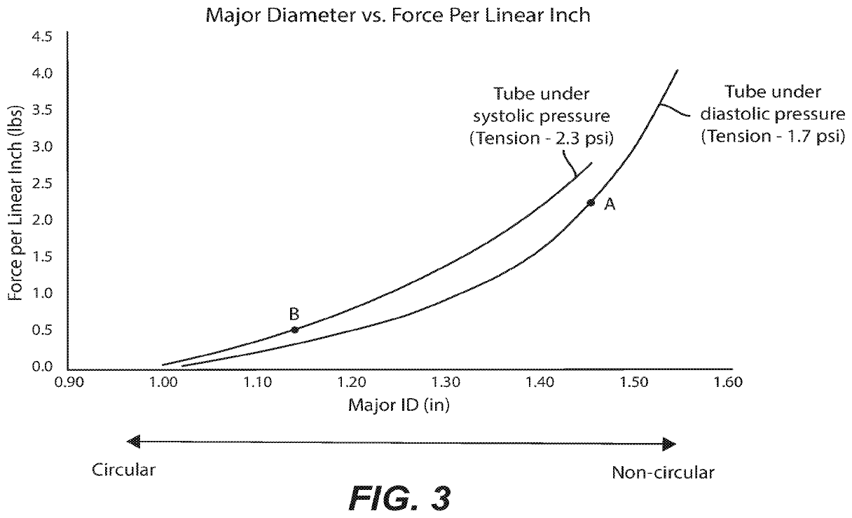

The inventors of the present application conducted an experiment to better understand the forces required for a device positioned within the aortic lumen (such as a stent) to change the cross sectional shape of the aorta from substantially circular to elongated under systolic and diastolic pressures. In the experiment, the aorta was approximated by a substantially cylindrical tube having a 1 inch diameter, which is similar to that of the aorta. As shown in FIG. 2A, two pairs of rigid rods were positioned at opposing sides of the tube. As shown in FIG. 2B, the pairs of rods were pulled in opposite directions to simulate forces exerted on the aortic wall by a stent having an elongated cross-sectional shape positioned within the aorta. While the force was applied, water was pumped through the tube at two pressures-88 mmHg (1.7 psi) to simulate diastolic pressure, and 120 mmHg (2.3 psi) to simulate systolic pressure. Force applied versus major diameter was recorded for both pressures as graphically depicted in FIG. 3.

The inventors hypothesized that deformation of the aorta between a substantially circular cross-sectional shape in systole and an ovular cross-sectional shape in diastole would improve compliance. The hypothesis was based on the premise that the greater the change in cross-sectional shape of the aorta between diastole and systole, the greater the change in cross-sectional area, and hence the greater the system compliance. However, if the stent exerts too much lateral force along the major diameter, the aorta may take the ovular cross-sectional shape in diastole but may not be able to achieve a cross-sectional shape in systole that is sufficiently circular to provide the change in volume necessary to meaningfully improve compliance. Conversely, if the stent is too flexible, the aorta will take a circular cross-sectional shape in systole, but may not be able to achieve a cross-sectional shape in diastole that is ovular enough to provide the change in volume necessary to meaningfully improve compliance. Without being bound by theory, it is believed that the optimal stent characteristics such that the stent would exhibit a lateral force of A (see FIG. 3B) at the given diameter and a force of B (see FIG. 3B) at the other given diameter.

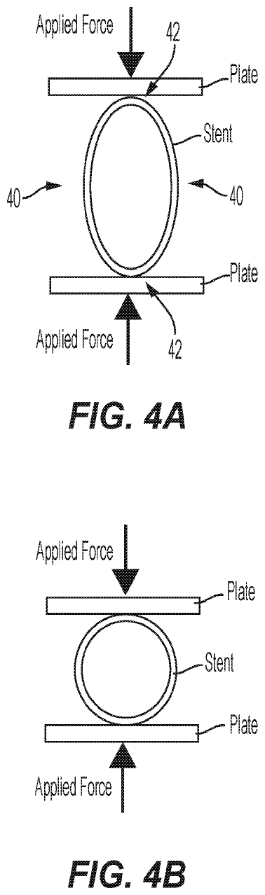

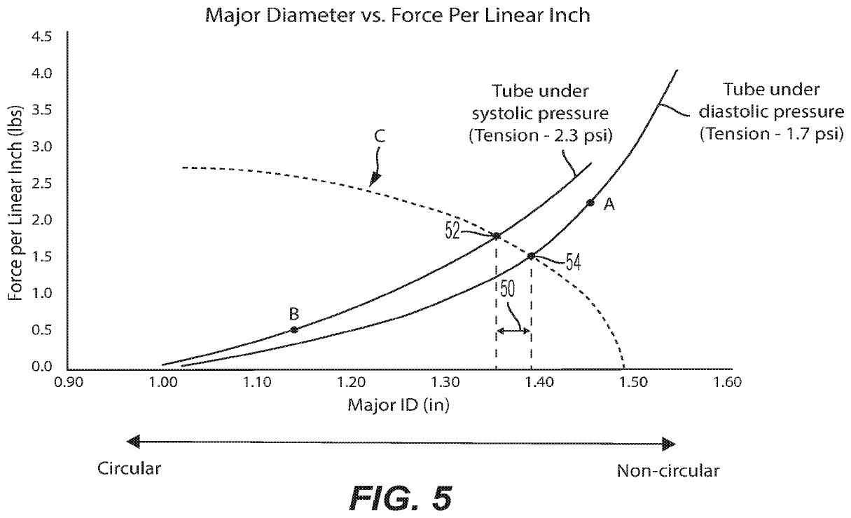

A second experiment was conducted by the inventors to better understand the forces exerted on a stent deployed within the aortic lumen by the aortic wall as the aortic wall pushes the stent from its heat set, ovular cross-sectional shape to a more circular cross-sectional shape. As shown in FIGS. 4A and 4B, a heat set, ovular stent was placed in a tensile tester between two force plates. The stent was compressed along its major axis to simulate the forces exerted on the short ends 42 of the stent by the aorta during systole. Force applied versus major diameter was recorded and is graphically represented by curve C in FIG. 5. In FIG. 5, curve C is shown superimposed on the diastolic and systolic plots of FIG. 3.

As shown in FIG. 5, as the ovular stent is compressed along its major diameter towards a more circular shape, the major diameter decreases but the force per linear inch increases. In other words, the more the aorta squeezes an ovular stent toward a more circular cross-sectional shape, the more the ovular stent resists. Because of this, curve C decreases in the direction of the non-circular shape and intersects the systolic pressure curve at point 52 and the diastolic pressure curve at point 54. The resulting difference 50 in major diameter between systole and diastole is minimal (less than 1/20 of an inch), thus providing little additional compliance.

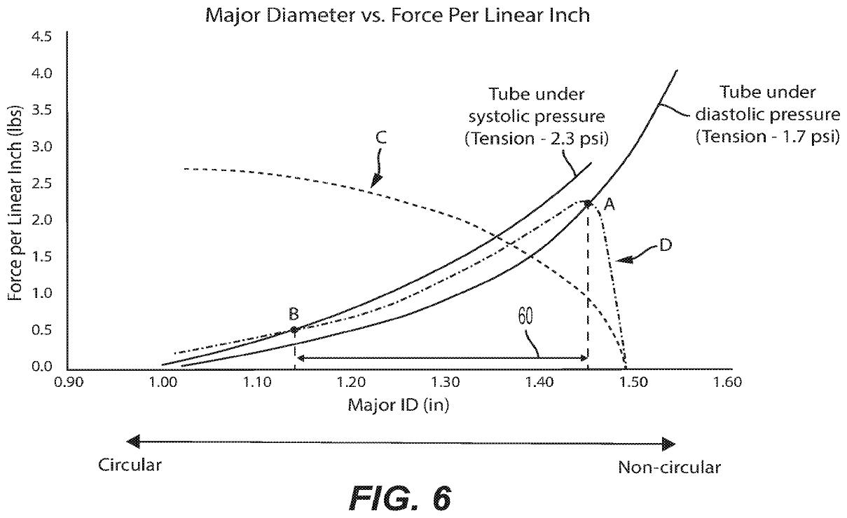

As detailed herein, the expandable structures of the present technology may have preloaded bend regions that exert a spring force that is generally constant when the expandable structure is compressed along the long dimension. Such a configuration enables the expandable structures of the present technology to follow curve D shown in FIG. 6, thereby providing a greater change in major diameter between diastole and systole and thus improved compliance.

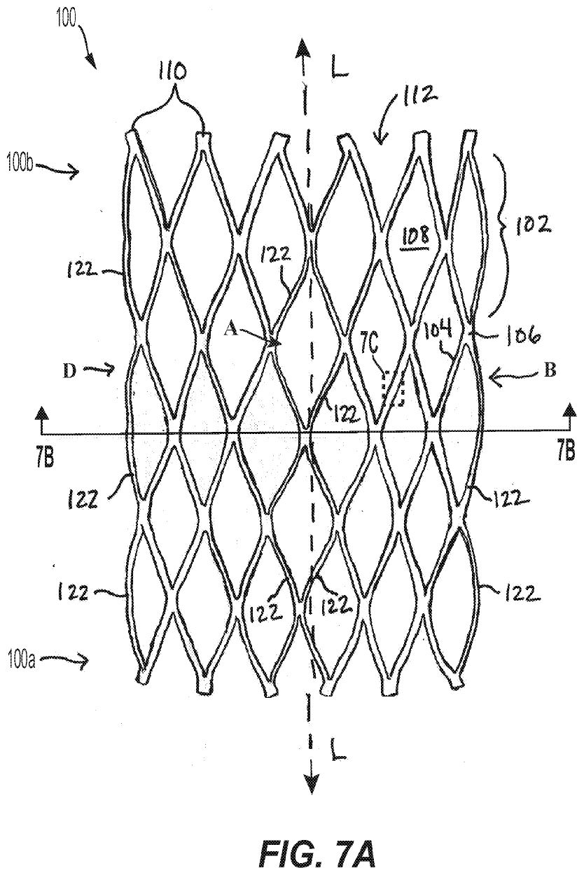

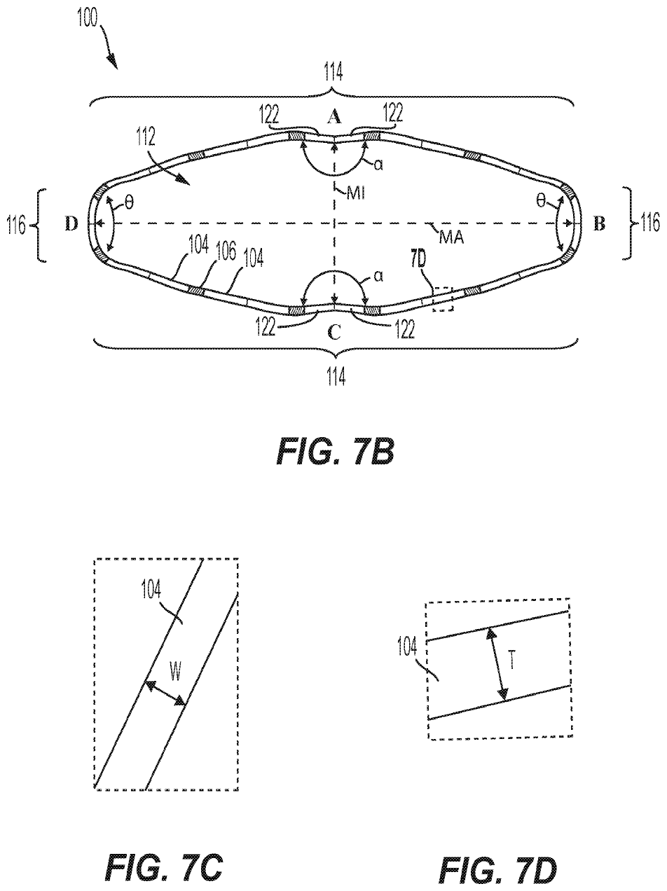

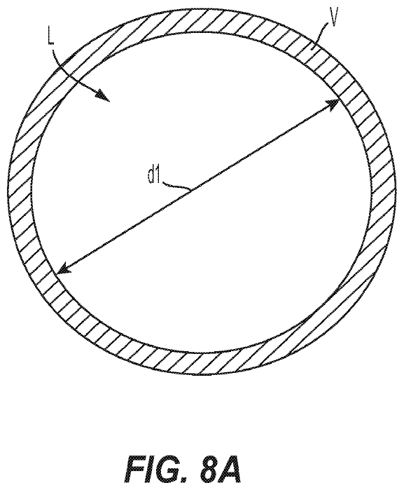

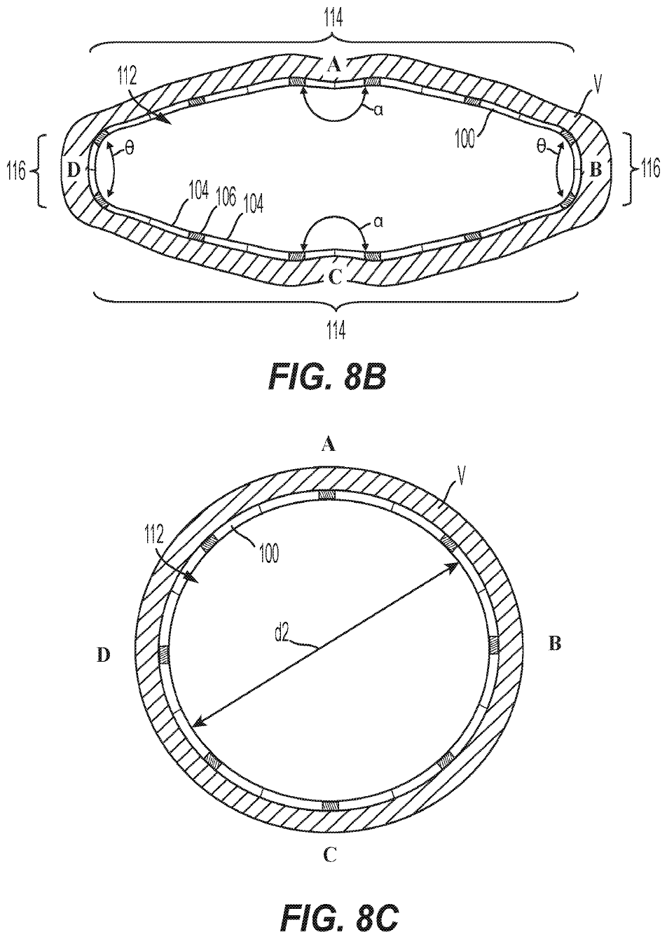

FIG. 7A is a side view of an expandable, generally tubular structure 100 configured in accordance with several embodiments of the present technology and having preloaded bend regions A and C. FIGS. 8A and 8B show the device of FIGS. 7A and 7B positioned within an artery during systole and diastole, respectively, in accordance with several embodiments of the present technology. As shown, the device 100 may be configured to be intravascularly delivered in a low-profile state to a treatment site within the lumen of an artery. The device 100 may be expanded at the treatment site, thereby assuming a pre-set, non-circular shape.

The device may comprise an expandable structure configured to be intravascularly positioned within the artery to improve arterial compliance. the aorta at a treatment site. The artery may have a substantially circular cross-sectional shape at the treatment site prior to deployment of the expandable structure, and wherein, when the expandable structure is in an expanded state and positioned in apposition with the arterial wall at the treatment site under diastolic pressure, the expandable structure forces the artery into a non-circular cross-sectional shape, wherein a cross-sectional area of the artery in the non-circular cross-sectional shape is less than a cross-sectional area of the artery in the substantially circular cross-sectional shape.

As described in further detail below, the device 100 can comprise a plurality of interconnected struts 104, each having a length, a width, and a thickness. As shown in the enlarged view of FIG. 7D, The thickness T can be measured as a dimension that is orthogonal to a central axis when the device 100 is considered in a tubular shape, or as a dimension that is orthogonal to a plane of the device 100 when represented as laid-flat. The length can be measured as a distance extending between ends of a strut, where the ends connect to another structure.

The minor diameter of the expandable structure may be as small as possible to maximize the volume change as it becomes round. However, the ends of the major diameter should not be sharp enough to cause damage to the aorta walls, and the minor diameter should be large enough that flow through the aorta is not impeded and there is no chance of thrombosis or other occlusion of the aorta. Therefore, the average minor diameter might be in the range of 6 mm-12 mm, and more preferably in the range of 8-10 mm. The expandable structure may increase compliance by 25-50 mL.

As the volume and pressure of the aorta increases, this will naturally tend to move the aortic walls facing the minor diameter of the stent outwards. As these walls move outwards, the aortic walls facing the major diameter of the stent will be pulled inwards, again since the circumference of the aorta is relatively fixed. As the major diameter of the stent is pulled inwards, the minor diameter will be pushed outwards, since deflecting the stent from an oval cross-section to a more rounded shape will require less force than it would take to compress and reduce the circumference of the stent. Therefore, the stent will become rounder as the aorta becomes rounder, and the stent walls and aorta walls should remain opposed throughout the cardiac cycle. This should lead to the stent healing into the wall over time.

The stent should be designed so that once deployed in the aorta, an aortic pressure somewhere between diastolic and systolic pressures is enough to distend the aorta from a flattened shape to a rounded shape. This will maximize the effect of the stent in increasing aortic compliance. Therefore with the stent in place, the aorta should preferably deform between an aortic pressure of 60 and 150 mmHg, and more preferably between 90 and 120 mmHg.

A rough calculation suggests that this device should provide enough compliance to have a significant effect. The typical stroke volume of the heart is 70 ml. Roughly ⅓ of that volume flows through the distal capillaries and organs in systole, leaving ⅔ or about 50 ml to flow during diastole.

If a perfectly round aorta with an inner diameter of 20 mm were flattened to a flat shape with a minor diameter of 8 mm, then it would have a major diameter of about 26.8 mm. The round aorta would have a cross-section of ˜314 square mm, and the flattened aorta would have an area of ˜201 square mm. Thus, each cm of flattened stent length would provide a potential accommodation of 1.13 ml. A 25 cm stent would provide potential accommodation of 28 mL. This would provide a significant additional compliance to the aorta, enough to provide at least half of the total compliance needed. This should significantly reduce systolic pressure and increase diastolic pressure, allowing the heart to do less work while at the same time improving tissue perfusion.

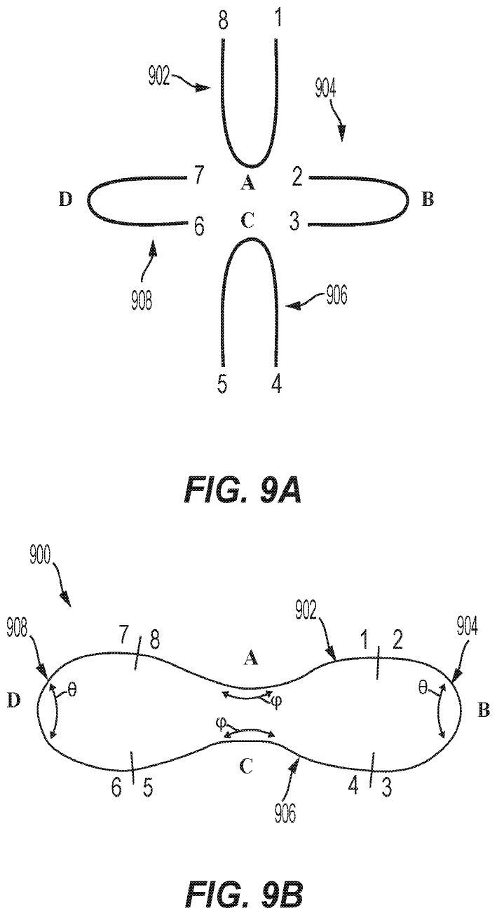

FIGS. 9A-11B depict a method for forming a preloaded device in accordance with several embodiments of the present technology. According to some embodiments, for example as shown in FIGS. 9A and 9B, a stent with pre-loaded bend regions can be formed from a plurality of strut regions 902-908. FIG. 9A shows an axial cross-sectional view of the plurality of strut regions 902-908. The plurality of strut regions can comprise a strut region 902 corresponding to a bend region A, a strut region 904 corresponding to a bend region B, a strut region 906 corresponding to a bend region C, and/or a strut region 908 corresponding to a bend region D. Each of the strut regions can include two ends and a bend region having a curvature therebetween. For example, strut region 902 can comprise a first end 1 and a second end 8 with bend region A therebetween. In some embodiments, the strut regions 902 and 906 can be oriented such that the bend regions A and C extend toward each other and the apices of strut region 902 extends away from the ends of strut region 906. Strut regions 904 and 908 can be oriented such that the bend regions B and D extend away from each other and the ends of strut region 904 extend toward the ends of strut region 908. A strut region can be heat treated to form the curvature of the strut region. In some embodiments, strut regions 902 and 906 have equivalent curvatures and strut regions 904 and 908 have equivalent curvatures.

In some embodiments, the stent can be formed from the plurality of strut regions by joining adjacent apices of neighboring strut regions, such as the stent depicted in FIG. 9B. For example, end 1 can be joined to end 2, end 3 can be joined to end 4, end 5 can be joined to end 6, and/or end 7 can be joined to end 8. The adjacent ends can be joined by laser-welding, resistance-welding, or another suitable method. FIG. 9B shows an end view of an example stent 900 formed from a plurality of strut regions 902-908. Each bend region can comprise an angle defining the degree of biasing of the bend region. In some embodiments, bend regions A and C can comprise an angle φ. Bend regions B and D can comprise an angle θ. In some embodiments, a thickness of the struts in a strut region can be based at least in part on a corresponding angle of the strut region. For example, struts in strut regions 904 and 908 can be narrower and/or thinner than struts in strut regions 902 and 906 because the angle φ of strut regions 904 and 908 is greater than the angle θ of strut regions 902 and 906.

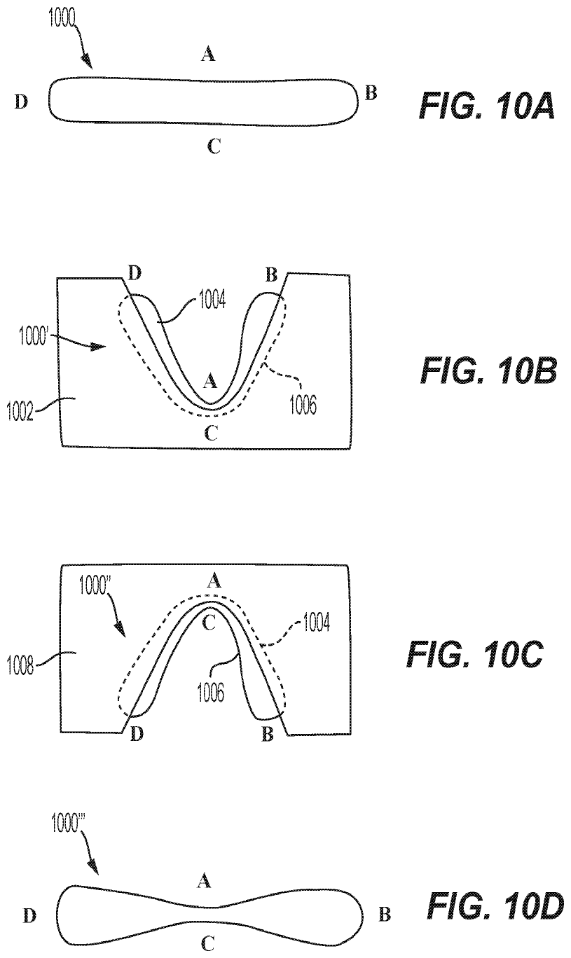

FIGS. 10A-10D depict a method for forming a preloaded device through heat treatment in accordance with several embodiments of the present technology. FIG. 10A shows an end view of a stent 1000 with a first cross-sectional shape having a long dimension and a short dimension that is orthogonal to the long dimension. In some embodiments, the first cross-sectional shape can be set by a heat treatment process. The stent 1000 can comprise strut regions with corresponding bend regions (e.g., bend region A, B, C, and/or D). According to some embodiments, one or more portions of the stent 1000 can be heat treated to create preloaded bend regions. For example, as depicted in FIG. 10B, the stent 1000′ can be attached to a heat treatment fixture 1002 such that a portion of the stent corresponding to bend region A 1004 is configured to be exposed to heat and a portion of the stent corresponding to bend region C 1006 is insulated. A heat treatment process can be used to set a preloaded shape of bend region A. FIG. 10C depicts the stent 1000″ attached to the heat treatment fixture 1002 such that a portion of the stent corresponding to bend region A 1004 is insulated and a portion of the stent corresponding to bend region C 1006 is configured to be heat treated. In some embodiments, one or more portions of the stent can be heat treated in the same process step. Alternatively, or in addition, portions of the stent can be heat treated individually and/or sequentially. As depicted in FIG. 10D, after heat treatment, the stent 1000′ can comprise a cross-sectional shape that is different from first cross-sectional shape of the stent 1000 before heat treatment (see FIG. 10A). For example, the stent 1000 can comprise a generally ovular cross-sectional shape before heat treatment, as depicted in FIG. 10A. The stent 1000′ can comprise a generally hourglass cross-sectional shape with preloaded bend regions A and C after heat treatment, as depicted in FIG. 10D.

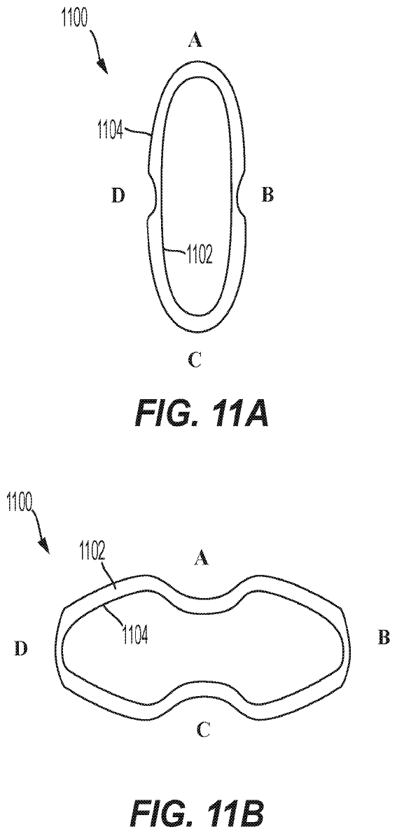

In some embodiments, a stent can be configured to have one cross-sectional shape in an initial state and another cross-sectional shape in an inverted state. For example, FIG. 11A shows an end view of a stent 1100 in an initial state with an inner surface 1102, and an outer surface 1104. The stent 1100 can comprise bend regions A, B, C, and D and an angle can be defined for each bend region. For example, FIG. 11A shows the stent 1100 with preloaded bend regions B and D. The stent 1100 can be inverted to bend the initial angles of each bend region by about 180 degrees and obtain a stent 1100 in an inverted state, as depicted in FIG. 11B. The stent 1100 in the inverted state can comprise different preloaded bend regions from the stent 1100 in the initial state. For example, as depicted in FIG. 11B, the stent 1100 in the inverted state can comprise preloaded bend regions A and C.

A cross-sectional shape of a stent as described herein can be defined by a perimeter of the stent. According to some embodiments, a cross-sectional shape can have a long dimension and a short dimension orthogonal to the long dimension. The stent can comprise first portions at either side of the long dimension and second portions at either side of the short dimension. Each of the first portions and the second portions can have a radius of curvature. In some embodiments, a radius of curvature of one first portion is the same as a radius of curvature of the other first portion. A radius of curvature of one second portion can be the same as a radius of curvature of the other second portion.

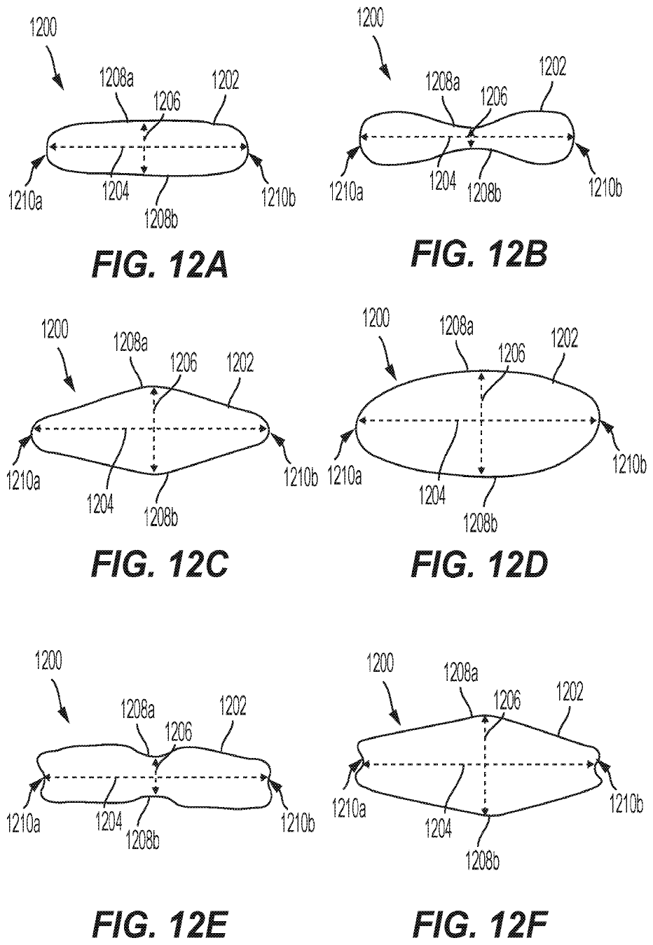

FIGS. 12A-12F show end views of several devices of the present technology with different cross-sectional shapes. FIG. 12A depicts an end view of a stent 1200 with a perimeter 1202 that defines a generally ovular cross-sectional shape with a long dimension 1204 and a short dimension 1206. The stent 1200 can comprise first portions 1208a and 1208b that are generally parallel to a long dimension of the stent and second portions 1210a and 1210b. First portions 1208a and 1208b can each be connected to opposite ends of second portions 1210a and 1210b to form the generally ovular cross-sectional shape. In some embodiments, first portions 1208a and 1208b can comprise preloaded bend regions that are biased toward a lumen of the stent (see FIG. 12B). The preloaded bend regions can be convex towards the lumen according to some aspects of the present technology. In some embodiments, the preloaded bend regions of first portions 1208a and 1208b are concave to the lumen, as shown in FIG. 12D. A radius of curvature of one or more portions can be adjusted based on a desired cross-sectional shape of a stent. For example, FIG. 12E depicts a stent 1200 with first portions 1208a and 1208b and second portions 1210a and 1210b that each have a radius of curvature that is greater than a radius of curvature of the stents depicted in FIGS. 12A-12C. As shown in FIG. 12C, in some embodiments, first portions 1208a and 1208b and second portions 1210a and 1210b can have preloaded bend regions biased towards the lumen of the stent 1200. In some embodiments, second portions 1210a and 1210b can have preloaded bend regions biased towards the lumen of the stent 1200 and first portions 1208a and 1208b can have preloaded bend regions biased away from the lumen of the stent 1200 (see FIG. 12F).

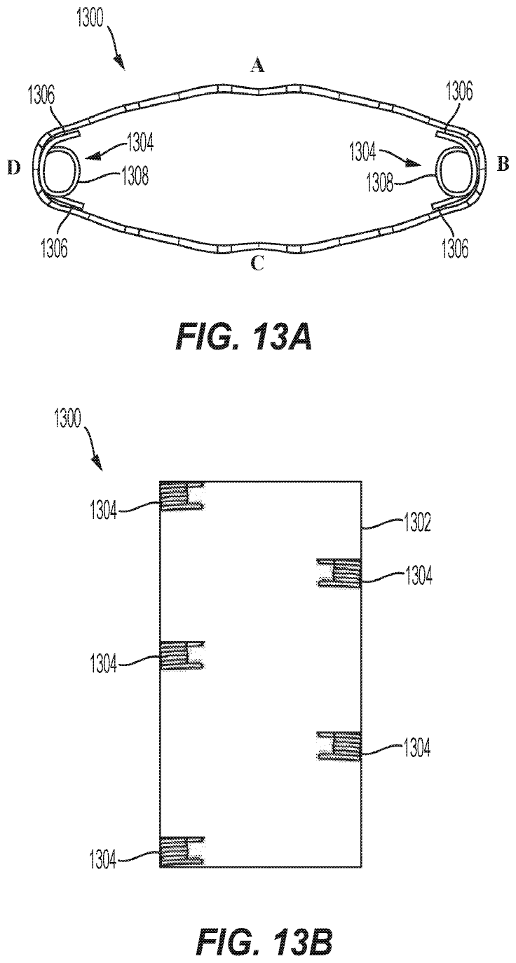

According to some embodiments of the present technology, a stent 1300 can be configured to include one or more torsion springs to facilitate a change in cross-sectional shape of the stent 1300 in response to a change in blood pressure, as depicted in FIGS. 13A and 13B. A torsion spring 1304 can have at least end portion 1306 positioned proximate to a first portion and/or a second portion of the stent 1300. For example, torsion springs 1304 are positioned proximate to the first portions of the stent 1300 corresponding to bend regions B and D in FIG. 13A. In some embodiments, an intermediate portion 1308 of the torsion spring 1304 can be configured to receive a force when an arterial wall exerts a force on the stent 1300 during systole. The force can be transferred from the intermediate portion 1308 to the end portion 1306 and the end portion 1306 can be configured to apply the force to a portion of the stent 1300 to facilitate a change in cross-sectional shape of the stent 1300 in response to the force exerted by the arterial wall. For example, the torsion springs 1304 proximate to bend regions B and D in FIG. 13A can facilitate second portions moving away from one another along a short dimension of the stent during systole. Torsion springs 1304 can be positioned along a length of a stent 1300 as depicted in FIG. 13B.

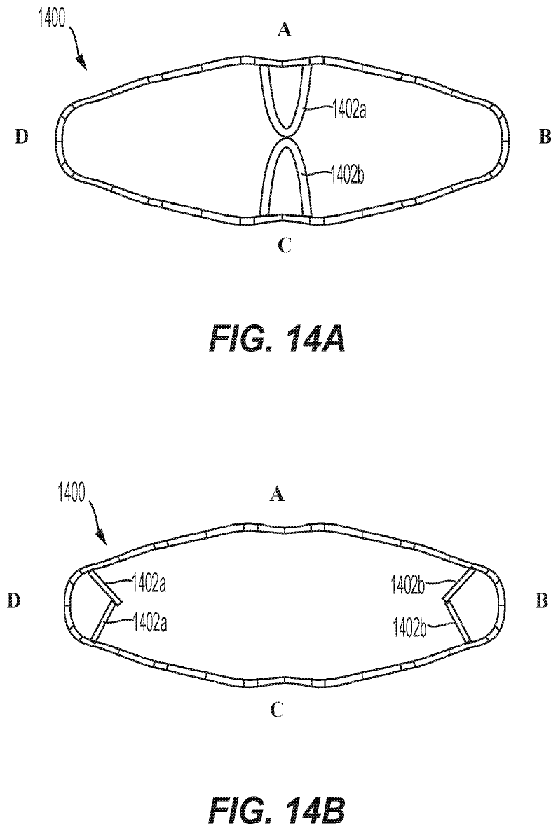

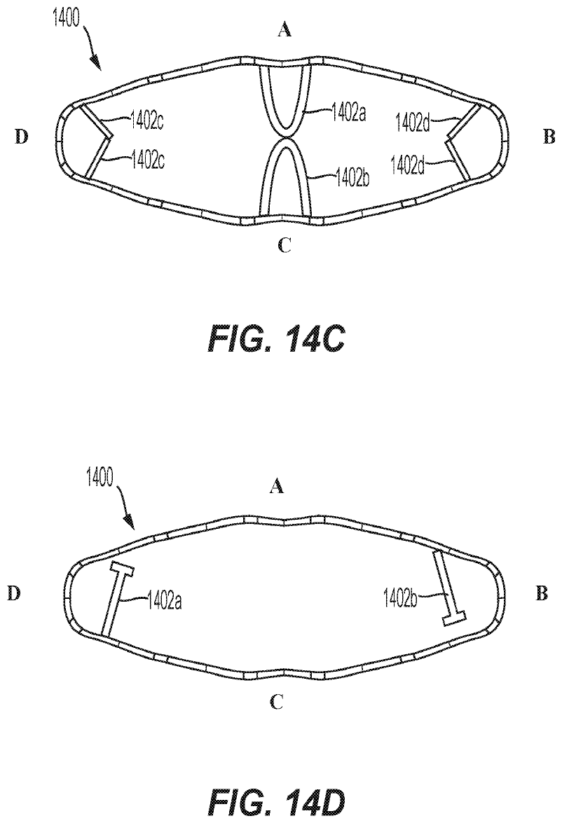

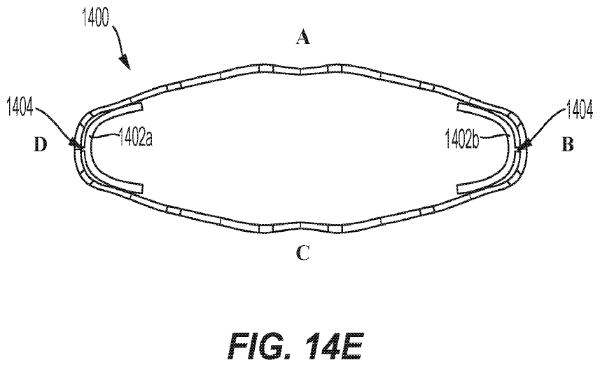

A stent in accordance with several embodiments of the present technology can include one or more supports within a lumen of the stent. For example, FIG. 14A shows an end view of a stent 1400 with a first support 1402a proximate to one first portion of the stent corresponding to bend region A and a second support 1402b proximate to another first portion of the stent corresponding to bend region C. As depicted in FIG. 14A, in some embodiments a first support 1402a can be configured to engage a second support 1402b to prevent a short dimension of the stent 1400 from decreasing below a minimum distance. According to some embodiments, for example in FIG. 14B, a stent can comprise first supports 1402a proximate one second portion of the stent corresponding to bend region D and a second support 1402b proximate another second portion of the stent corresponding to bend region B. The first and second supports 1402a and 1402b can be configured to extend into the lumen of the stent 1400. In some embodiments, a stent 1400 can comprise supports 1402a and 1402b proximate first portions of the stent and supports 1402c and 1402d proximate second portions of the stent, as shown in FIG. 14C. According to some embodiments, first and second supports 1402a and 1402b can comprise a first end portion attached to the stent and a second end portion spaced apart from an opposing portion of the stent, as depicted in FIG. 14D. FIG. 14E shows an axial cross-sectional view of a stent 1400 with C-shaped first and second supports 1402a and 1402b positioned proximate to second portions of the stent 1400. The first and second supports 1402a and 1402b can include a projection 1404 positioned at an apex of the support configured to attach to the stent 1400. The projection 1404 can permit a radius of curvature of bend regions B and D of the stent 1400 to increase in response to forces exerted by the arterial wall, while the first and second supports 1402a and 1402b prevent a short dimension of the stent from decreasing below a minimum distance.

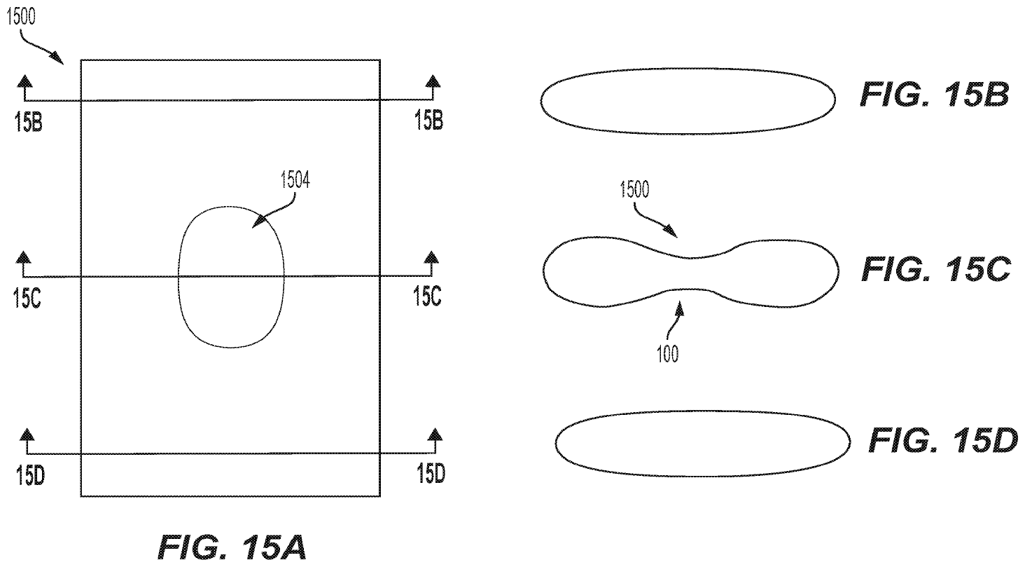

According to some embodiments, for example as shown in FIGS. 15A-15D, a stent 1500 can comprise end portions 15B and 15D with one cross-sectional shape and an intermediate portion 15C with another cross-sectional shape. For example, as shown in FIGS. 15B and 15D, the end portions can comprise a generally ovular cross-sectional shape while the intermediate portion can comprise a generally hourglass cross-sectional shape. In some embodiments, one or more portions of a stent can comprise one cross-sectional shape and one or more remaining portions can comprise another cross-sectional shape. Alternatively, or in addition, all portions of a stent can comprise the same cross-sectional shape and/or all portions of a stent can comprise different cross-sectional shapes.

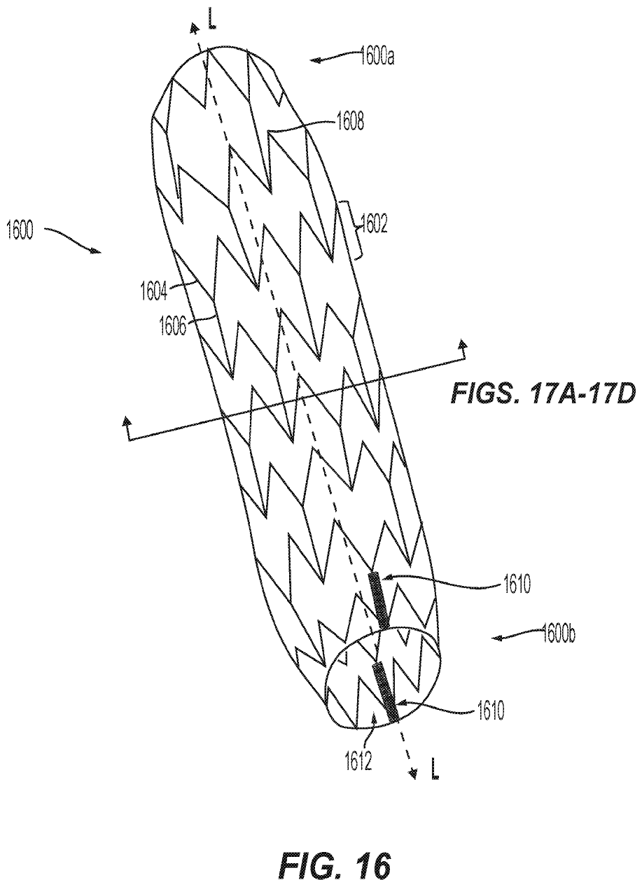

The present technology relates to devices, systems, and methods for treating blood vessels. In particular, the present technology relates to devices, systems, and methods for treating arteries. In some embodiments, for example, the devices of the present technology are configured to increase aortic compliance. A device of the present technology is an expandable structure 1600, for example as shown in FIG. 16. The expandable structure 1600 can be configured to have a low-profile state for delivery of the device to a treatment site within an artery and/or an expanded state corresponding to a device that has been deployed within an artery. The expandable structure 1600 can comprise a first end portion 1600a, a second end portion 1600b, an intermediate portion, and a length extending between the first and second end portions 1600a, 1600b along a longitudinal axis L (see FIG. 16) of the expandable structure 1600. According to some embodiments, the expandable structure 1600 has a non-circular cross-sectional shape.

A device of the present technology can comprise an expandable structure 1600 comprising a plurality of strut regions 1602 extending circumferentially about the expandable structure 1600. Each strut region 1602 can comprise a plurality of struts 1604 and a plurality of apices 1608. In some embodiments, the longitudinal struts 1606 can extend between adjacent strut regions 1602. A lumen 1612 of the expandable structure 1600 can be defined by the struts 1604. In some embodiments, the strut regions 1602 can comprise continuous circumferential rings as depicted in FIG. 16. The struts 1604 of a strut region 1602 can be connected at apices 1608 such that the struts 1604 are disposed in a zig-zag pattern to facilitate radial compression and expansion of the expandable structure 1600. The struts 1604 of a strut region 1602 can be connected in a pattern to enhance longitudinal flexibility of the expandable structure 1600. The stent may have radiopaque markers positioned at the first end portion, at the second end portions, and/or therebetween, as shown in FIG. 16. Radiopaque markers 1610 can be positioned on the expandable structure 1600 to facilitate visualization of the device during delivery. For example, the expandable structure 1600 can include radiopaque markers located on anterior and posterior portions of the stent to visualize the device with a direct anterior-posterior fluoroscopy view.

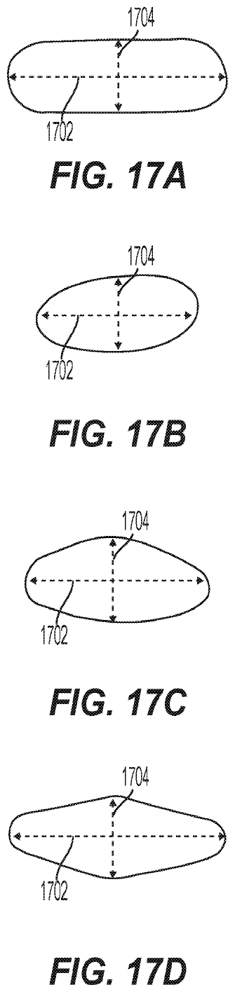

According to some embodiments, for example as shown in FIGS. 17A-17D, the expandable structure can have a non-circular cross-sectional shape. The cross-sectional shape can have a long dimension 1702 and a short dimension 1704. In some embodiments, the short dimension 1704 can be between about 6 mm and 12 mm and the long dimension 1702 can be between about 15 mm and 40 mm. The non-circular cross-sectional shape can have parallel major walls as shown in FIG. 17A, slightly curved walls as shown in FIG. 17B, a generally oval shape as shown in FIG. 17C, a generally rhomboidal shape as shown in FIG. 17D, or a variation of these shapes. The cross-sectional shape of the expandable structure 1600 can be configured such that a wall of an artery conforming to the cross-sectional shape of the expandable structure 1600 has the same cross-sectional shape as the expandable structure 1600. In some embodiments, the cross-sectional shape of the expandable structure 1600 can be configured to flatten a cross-sectional shape of an artery in an anterior-posterior direction, a lateral direction, and/or at an oblique angle. An angle can be selected to minimize any impact on surrounding organs, structures, and/or branch vessels. In some embodiments, the angle varies over a length of the stent. In some embodiments, an end portion of the expandable structure 1600 comprises a generally circular cross-sectional shape and an intermediate portion of the stent between the end portions comprises a generally non-circular cross-sectional shape, as shown in FIG. 16. A generally circular cross-sectional shape of end portions of the expandable structure 1600 can facilitate a smooth transition in cross-sectional shape between a portion of an artery conforming to the expandable structure 1600 and a portion of the artery without the expandable structure 1600. Additionally, or alternatively, a stiffness of the end portions of the expandable structure 1600 can be less than a stiffness of the intermediate portion of the expandable structure 1600 to facilitate a smooth transition between various portions of the artery.

A device of the present technology can be configured to be positioned at a treatment site within a lumen of an artery, such as an aorta. An expandable structure 1600 of the device can comprise a low-profile state for delivery of the device to the treatment site and/or an expanded state with a non-circular cross-sectional shape for maintaining a cross-sectional shape of the artery at the treatment site. In the expanded state, the expandable structure 1600 can be configured to be positioned in apposition with an arterial wall at the treatment site. Under diastolic pressure, the expandable structure 1600 can cause the arterial wall to conform to the non-circular cross-sectional shape of the expandable structure 1600. A cross-sectional area based on the non-circular cross-sectional shape of the artery can be less than a cross-sectional area of a circular cross-sectional shape of the artery. For example, the expandable structure 1600 can comprise a long dimension and a short dimension, and the expandable structure 1600 can comprise first portions at either end of the long dimension and second portions at either end of the short dimension. When positioned within the artery in the expanded state, the expandable structure 1600 can cause a radius of curvature of portions of the arterial wall proximate to the second portions of the expandable structure 1600 to increase. By decreasing the cross-sectional area of the artery during diastole, the artery can undergo a greater change in volume throughout a cardiac cycle. Reducing the cross-sectional area of the artery can thereby increasing a compliance of the arterial system without stretching the arterial wall. Such increase in compliance can be advantageous in arteries with reduced capacity to stretch (e.g., arteries with calcification).

During systole, blood pressure within an artery can increase and cause the artery to deform. As the volume and pressure of an artery increases during systole, the artery can exert forces on second portions of the expandable structure 1600. In response to the exerted forces, opposing second portions of the expandable structure 1600 can be configured to move toward each other and opposing first portions of the expandable structure 1600 can be configured to move away from each other. As a result, the expandable structure 1600 and artery can assume a second cross-sectional shape and a second cross-sectional area. In some embodiments, the second cross-sectional shape is generally circular, and the second cross-sectional area is generally greater than a cross-sectional area of the first cross-sectional shape. The change in cross-sectional shape can thereby absorb and reduce energy transmitted to the arterial system from the left ventricle during systole. In some embodiments, a circumference of the artery and/or the expandable structure 1600 does not change during systole.

In some embodiments, it may be advantageous for the expandable structure 1600 to be configured to assume a second cross-sectional shape different from a first cross-sectional shape at a predetermined pressure or range of pressures. For example, a device configured to be placed in an aorta can be configured to expand at an aortic pressure between diastolic and systolic pressure to increase the compliance of the aorta. The expandable structure 1600 can be configured to deform between an aortic pressure of about 60 and about 150 mmHg. In some embodiments, the expandable structure 1600 can be configured to deform between an aortic pressure of about 90 and about 120 mmHg.

According to some embodiments, the device is configured to be position in a portion of the aorta such as the ascending aorta, the aortic arch, the descending thoracic aorta, the abdominal aorta, or even the iliac arteries. One or more devices can be deployed in multiple sections of the aorta. A size, shape, or taper of the device can be determined based on the portion of the aorta that the device is configured to be positioned within. During deployment of the device, it may be advantageous to include a distal filter to capture emboli. In some embodiments, the expandable structure 1600 of the device includes long struts to permit fluid flow to a branching artery such as a celiac artery, a renal artery, a mesenteric artery, a vertebral artery, a brachiocephalic artery, a carotid artery, and/or a subclavian artery.

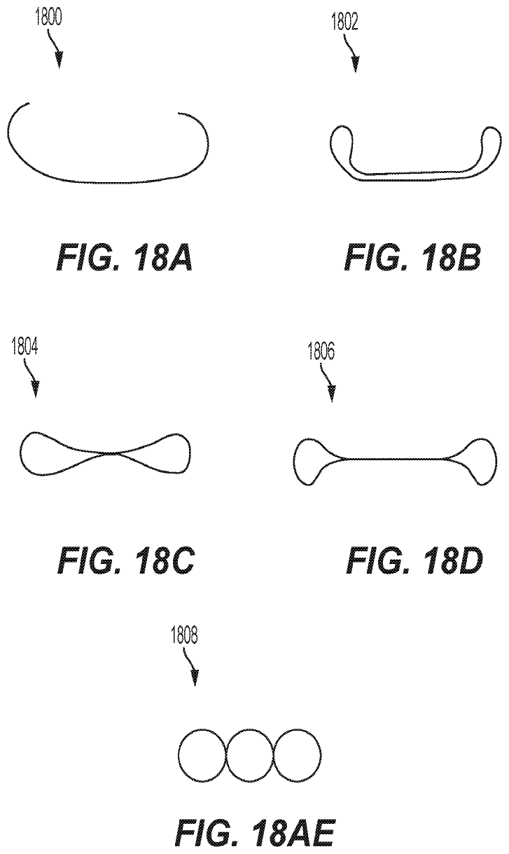

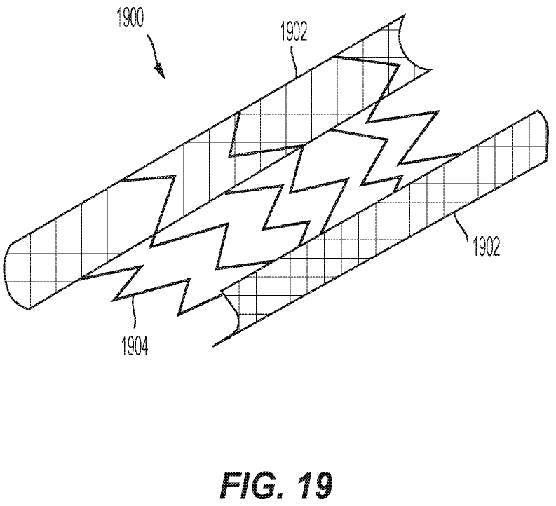

According to some embodiments of the present technology, an expandable structure is configured to maintain a non-circular cross-sectional shape of an artery during diastole and expand to assume a circular cross-sectional shape during systole. In some embodiments, the expandable structure can have a non-circumferential design. Alternative, non-circumferential cross-sectional shapes are shown in FIGS. 18A-18E. The expandable structure can comprise a C-shaped cross-sectional shape 1800 and 1802, an hourglass cross-sectional shape 1804, a dog-bone cross-sectional shape 1806 and/or a cross-sectional shape comprised of multiple round strut regions 1808. In some embodiments, an expandable structure 1900 can have multiple curved sections 1902 configured to engage an arterial wall and one or more support struts 1904 configured to maintain a distance between the curved sections 1902, as shown in FIG. 19.

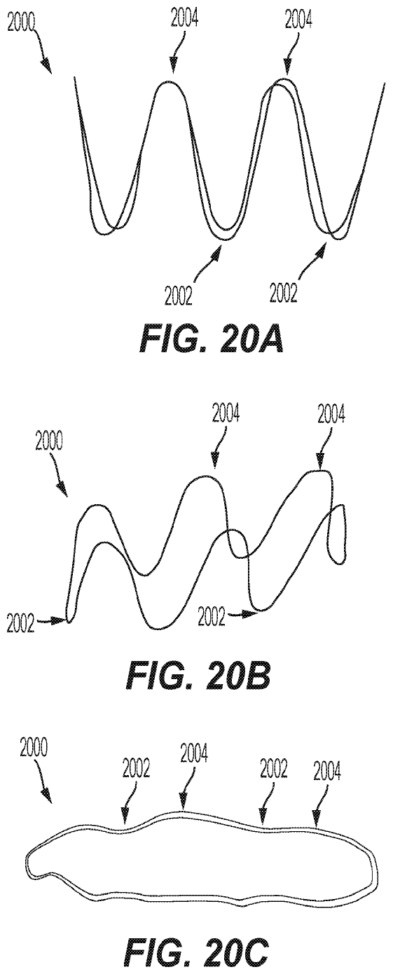

In some embodiments, an expandable structure may be formed by laser-cutting a desired pattern into a tubular sheet of material. In certain embodiments, the expandable structure may be initially formed as a flat sheet of material having a pattern of struts. The struts may be formed by depositing a thin film on a flat surface in the desired pattern, or by laser-cutting a desired pattern into the flat sheet of material. The flat pattern may then be curled up into a generally tube-like shape such that the longitudinal edges of the flat pattern are positioned adjacent to or in contact with one another. The longitudinal edges can be joined (e.g., via laser welding) along all or a portion of their respective lengths. In some embodiments, the struts may be formed by depositing a thin film on the surface of a tubular frame in a desired pattern (e.g., via thin film deposition, vapor deposition, or combinations thereof). As depicted in FIGS. 20A-20C, in some embodiments an expandable structure can comprise strut regions 2000 formed of a single, continuous wire. The strut regions 2000 can comprise a plurality of struts and a plurality of apices 2002 and 2004. Apices of one strut region 2000 can be connected to apices of another adjacent strut region 2000 (e.g., via laser welding) to form an expandable structure comprising multiple strut regions 2000.

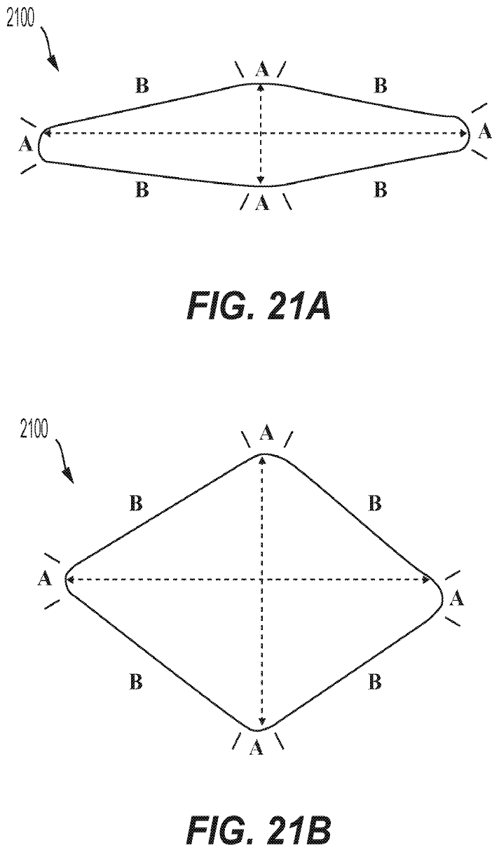

In some embodiments, it may be advantageous to for an expandable structure to be configured to remain in direct contact with a portion of an arterial wall throughout a full cardiac cycle. To maximize contact of an expandable structure with an arterial wall throughout the cardiac cycle, in some embodiments the expandable structure has resilient bend regions configured to expand under systolic blood pressure such that a cross-sectional area of the expandable structure changes throughout the expansion and compression of a circumference of the stent is minimized (see FIGS. 21A and 21B). According to some embodiments of the present technology, an expandable structure 2100 can have a first cross-sectional area associated with a first, non-circular cross-sectional shape of the expandable structure 2100 (see FIG. 21A) and a second cross-sectional area associated with a second, expanded cross-sectional shape (see FIG. 21B). The second cross-sectional shape can be configured to maximize contact with an arterial wall throughout a cardiac cycle.

As shown in FIGS. 21A and 21B, an expandable structure 2100 can have a generally rhomboidal cross-sectional shape with resilient bend regions A at either side of a short dimension of the cross-sectional shape and/or either side of a long dimension of the cross-sectional shape. Generally straight regions B can extend between neighboring bend regions A. A stiffness of a straight and/or bend region can be based on a width, a thickness, a length, and/or a material property of struts of the region. For example, the generally straight regions B can be configured to be stiffer than the generally bent regions A by using wider, thicker, and/or shorter struts. The generally bent regions A can be configured to be less stiff than the generally straight regions B by using, less wide, thinner, and/or longer struts. A material the struts are formed of can be selected based on a desired stiffness of the portions. Based on relative stiffnesses of the bent and straight regions A and B, the bent regions A can bend under systolic pressure in response to forces exerted on the expandable structure 2100 by the arterial wall. A pattern of strut regions can be selected to prevent crack formation at the bent regions A.

According to some aspects of the present technology, a flexible delivery catheter and/or catheter system can be used to deliver the device to an artery. The delivery catheter can be inserted into a patient's femoral artery, carotid artery, and/or any other vessel suitable for percutaneous or vascular surgical techniques. In some embodiments, the delivery catheter can include a guidewire lumen and can be configured to be advanced over a guidewire. The delivery catheter can have a tapered distal end to mitigate traumatic injury to a vessel from advancement of the catheter. An expandable structure of a device of the present technology can be compressed to assume a low-profile state by a cover sleeve. In some embodiments, the cover sleeve can be withdrawn to allow the expandable structure to expand from the low-profile state to the expanded state. The cover sleeve can be advanced over the stent after having been previously withdrawn to compress the expandable structure to the low-profile state for repositioning and/or retrieval.

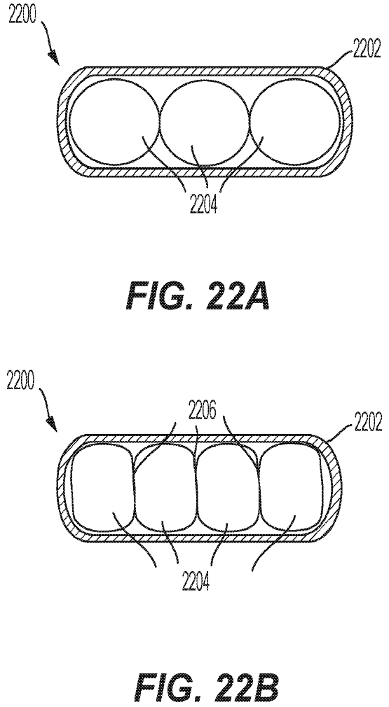

In any of the embodiments detailed herein, the device structure may be self-expanding. A self-expanding device can be formed of a shape memory alloy such as nitinol, for example. In some embodiments, the device can be balloon-expandable and formed of a stainless-steel alloy, a cobalt-chromium alloy, and/or other similar materials. Balloon catheters for expanding balloon-expandable devices typically have a circular volume when inflated. In some embodiments, it may be advantageous to configure a balloon catheter comprising a non-circular volume when inflated to maintain a corresponding non-circular cross-sectional shape of the expandable structure of the device. FIGS. 22A and 22B show example balloons configured for use in a balloon catheter to expand a device with a non-circular cross-sectional shape. For example, as depicted in FIG. 22A, a balloon comprising an ovular inflated volume can comprise a plurality of tubular balloons 2202 joined by a balloon wall 2200 surrounding the plurality of tubular balloons 2202. A balloon with an ovular inflated volume can comprise a balloon wall 2200 surrounding a plurality of chambers 2104 separated by chamber walls 2106.

In some embodiments, a device in accordance with the present technology may be coated with an anti-proliferative and/or an anti-thrombotic coating to prevent thrombosis of the treatment site and/or a healing response that increases a stiffness of the artery being treated. The device can include a coating, surface texture, and/or covering member disposed on a radially outer surface and/or a radially inner surface of the expandable structure. For example, a covering member comprising polyester fibers can be disposed on a radially outer surface of the expandable structure to promote ingrowth of arterial wall tissue into the expandable structure. Ingrowth can be advantageous to mitigate device fatigue and/or aneurysm formation in the arterial wall. Additionally, the device can be configured to promote ingrowth such that the device is incorporated into arterial and configured to reduce the stress experienced by the arterial wall throughout the cardiac cycle. In some embodiments, the device comprises a plurality of cells in the expandable structure to permit fluid flow in branch vessels. In some embodiments, a device can be sized to be slightly larger than an artery of the treatment site such that one or more portions of an arterial wall are in contact with the device for a desired portion of the cardiac cycle.

1. A device for treating an artery of a human patient, the device comprising:
an expandable structure configured to be intravascularly positioned within a lumen of the artery at a treatment site, wherein the artery has a substantially circular cross-sectional shape at the treatment site prior to deployment of the expandable structure therein, and wherein, when the expandable structure is in an expanded state and positioned in apposition with the arterial wall at the treatment site under diastolic pressure, the expandable structure forces the artery into a non-circular cross-sectional shape, wherein a cross-sectional area of the artery in the non-circular cross-sectional shape is less than a cross-sectional area of the artery in the substantially circular cross-sectional shape.

2. The device of claim 1, wherein, when the expandable structure is in the expanded state and in apposition with the arterial wall at the treatment site under systolic pressure, the arterial wall deforms in response to the increase in blood pressure towards a more circular cross-sectional shape, thereby deforming the expandable structure as well.

3. The device of claim 2, wherein a cross-sectional area of the artery in the more circular cross-sectional shape is greater than a cross-sectional area of the artery in the non-circular cross-sectional shape.

4. The device of claim 1, wherein the non-circular cross-sectional shape is one of an oval, an ellipse, a rhomboid, or an hourglass.

5. The device of claim 1, wherein the expandable structure comprises two relatively rigid linear elements with curved cross-sections, separated by one or more springs which hold them apart.

6. The device of claim 5, wherein a preload and a geometry of the springs cause a force holding the linear elements apart to decrease as the two linear elements are pressed closer together.

7. The device of claim 1, wherein the artery is the aorta.

8-11. (canceled)

12. The device of claim 1, wherein the expandable structure comprises a superelastic material.

13. The device of claim 1, wherein the expandable structure is non-circular in the expanded state.

14. The device of claim 1, wherein the expandable structure is non-circular when positioned in the arterial lumen in the expanded state.

15-52. (canceled)

53. A device for treating an artery, the device comprising:
an expandable structure comprising a first elongated element, a second elongated element, and a spring extending between the first and second elongated elements, the expandable structure being configured to be intravascularly positioned within a lumen of the artery at a treatment site such that the first elongated element is positioned in apposition with the arterial wall at a first position about a circumference of the arterial wall, the second elongated element is positioned in apposition with the arterial wall at a second position about the circumference of the arterial wall spaced apart from the first position, and the expandable structure exerts a radially outward force on the arterial wall, wherein, in response to an increase in pressure within the arterial lumen, a distance between the first and second elongated elements decreases and the radially outward force decreases and wherein, in response to a decrease in pressure within the arterial lumen, the distance and the radially outward force increase.

54. The device of claim 53, wherein, under diastolic pressure, the expandable structure forces the artery into a cross-sectional shape having a cross-sectional area less than a cross-sectional area of the artery prior to deployment of the expandable structure therein.

55. The device of claim 54, wherein under systolic pressure, the arterial wall deforms the expandable structure such that the artery assumes a cross-sectional shape having a cross-sectional area greater than the cross-sectional area of the cross-sectional shape of the artery under diastolic pressure.

56. The device of claim 55, wherein the cross-sectional shape of the artery under systolic pressure is substantially circular and the cross-sectional shape of the artery under diastolic pressure is substantially oblong.

57. The device of claim 53, wherein, the expandable structure is configured to be positioned within the arterial lumen such that the first and second elongated elements extend from first ends to second ends along a longitudinal axis of the artery.

58. The device of claim 53, wherein at least one of the first elongated element or the second elongated element has a curved cross-sectional shape.

59. The device of claim 53, wherein the expandable structure has circumferentially discontinuous cross-sectional shape.

60. The device of claim 53, wherein the spring extends from a first end at the first elongated element to a second end at the second elongated element in a zig-zag pattern.

61. The device of claim 60, wherein the spring is a first spring, the expandable structure further comprising a second spring a first end at the first elongated element to a second end at the second elongated element in a zig-zag pattern.

62. The device of claim 53, wherein the artery is an aorta of the patient.
